(19) United States
(12) Patent Application Publication
(10) Pub. No.: US 2026/0240649 A1
(43) Pub. Date: Aug. 20, 2026

(54) FRAMES FOR PROSTHETIC VALVES

(71) Applicant: Edwards Lifesciences Corporation, Irvine, CA (US)

(72) Inventors: Tamir S. Levi, Zikhron Yaakov (IL); Yulia Mordukhaev, Ramat Gan (IL)

(73) Assignee: Edwards Lifesciences Corporation, Irvine, CA (US)

(21) Appl. No.: 19/646,431

(22) Filed: Apr. 13, 2026

Related U.S. Application Data
(63) Continuation of application No. PCT/US2024/052319, filed on Oct. 22, 2024.
(60) Provisional application No. 63/592,454, filed on Oct. 23, 2023.

(51) Int. Cl. A61F 2/24

(52) U.S. Cl. CPC A61F 2/2418; A61F 2220/0075; A61F 2250/0036; A61F 2250/0037

(57) ABSTRACT

The present disclosure relates to prosthetic valves with enlarged cells along an upper cell row. In an example, a prosthetic valve comprises a frame that can include a first, second, and third rungs of angled struts, and a first cell row comprising a plurality of axial frame members extending between the first rung and the second rung of angled cells, wherein each cell of the first cell row is defined between immediately adjacent axial frame members and angled struts of the first and second rungs. In some examples, at least three angled struts along any of the first rung and/or second rung can extend between immediately adjacent axial frame members.

### CROSS-REFERENCE TO RELATED APPLICATIONS

This application is a continuation of International Application No. PCT/US2024/052319, filed October 22, 2024, which claims the benefit of U.S. Provisional Application No. 63/592,454, filed October 23, 2023, which is incorporated by reference herein.

### FIELD

The present disclosure relates to prosthetic valves, and in particular, to frames of prosthetic heart valves than can include upper rows of enlarged cells.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, towards the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

A prosthetic valve includes a leaflet assembly coupled to a support frame that includes intersecting struts, defining rows of closed cells. Some of these cells may be facing the coronary ostia in the case of aortic valve replacement. In some instances, a patient may require implantation of a coronary stent or other procedure that requires access to a coronary artery after prosthetic valve implantation. For such instances, a physician may need to access the coronary artery through an opening defined by a cell facing the coronary ostium. Accordingly, a need exists for improved prosthetic valves designed to provide adequate access to the coronary arteries therethrough.

The present disclosure is directed towards prosthetic valve comprising an expandable frame having several rows of cells, wherein an upper row which is closer to the outflow end of the valve can include cells which are larger than those of other cell rows.

According to some aspects of the disclosure, there is provided a prosthetic valve comprising a frame movable between a radially compressed state and a radially expanded state. The frame extends between an inflow end and an outflow end. The frame comprises a first rung of angled struts interconnected at upper junctions and lower junctions of the first rung, a second rung of angled struts interconnected at upper junctions and lower junctions of the second rung, and a third rung of angled struts interconnected at upper junctions and lower junctions of the first rung. The second rung is distal to the first rung. The third rung is distal to the second rung.

The frame further comprises a first cell row and a second cell row, each cell row comprising a plurality of cells. The first cell row comprises a plurality of axial frame members. Each axial frame member is connected at an upper end thereof to the first rung, and at a lower end thereof to the second rung. At least one cell of the first cell row is defined between two of the axial frame members, and a plurality of angled struts of the first and second rungs extending between the two axial frame members.

In some examples, each cell of the second cell row defined between two angled struts of the second rung and two angled struts of the third rung.

In some examples, at least one cell of the first cell row comprises at least three angled struts of the second rung.

In some examples, at least some of the cells of the first cell row are wider, in the

In some examples, the upper junctions of the first rung are outflow apices of the frame.

In some examples, the axial frame members are parallel to each other.

In some examples, the axial frame members are parallel to a central axis of the frame.

In some examples, the second cell row is closer to the inflow end than the first cell row.

In some examples, the angled struts of each of the first rung, the second rung, and the third rung, are circumferentially arranged in a zig-zagged pattern.

In some examples, the axial frame members comprise a plurality of axial struts and a plurality of commissure supports.

In some examples, the commissure support is configured to support a commissure of a valvular structure that can be mounted in the frame.

In some examples, the commissure window comprises a window opening between two window struts of the commissure window.

In some examples, at least one cell of the first cell row spans a width of one and a half cells of the second cell row.

In some examples, the second rung comprises three angled struts extending between immediately adjacent axial frame members along the first cell row.

In some examples, the first rung comprises three angled struts extending between immediately adjacent axial frame members along the first cell row.

In some examples, the commissure supports are attached at their upper ends to the upper junctions of the first rung, and are attached at their lower ends to lower junctions of the second rung.

In some examples, the axial struts are attached at their upper ends to the lower junctions of the first rung, and are attached at their lower ends to upper junctions of the second rung.

In some examples, wherein the commissure supports are longer than the axial struts.

In some examples, the commissure supports are attached at their upper ends to the lower junctions of the first rung, and are attached at their lower ends to upper junctions of the second rung.

In some examples, the axial struts are attached at their upper ends to the upper junctions of the first rung, and are attached at their lower ends to lower junctions of the second rung.

In some examples, the axial struts are longer than the commissure supports.

In some examples, the commissure supports are attached at their upper ends to the lower junctions of the first rung, and are attached at their lower ends to lower junctions of the second rung.

In some examples, the axial struts are attached at their upper ends to the upper junctions of the first rung, and are attached at their lower ends to upper junctions of the second rung.

In some examples, the commissure supports are attached at their upper ends to the upper junctions of the first rung, and are attached at their lower ends to upper junctions of the second rung.

In some examples, the axial struts are attached at their upper ends to the lower junctions of the first rung, and are attached at their lower ends to lower junctions of the second rung.

In some examples, the axial struts and the commissure supports have equal lengths.

In some examples, the first rung comprises two angled struts extending between immediately adjacent axial frame members along the first cell row.

In some examples, the commissure supports are attached at their upper ends to the lower junctions of the first rung, and are attached at their lower ends to lower junctions of the second rung.

In some examples, the axial struts are attached at their upper ends to the lower junctions of the first rung, and are attached at their lower ends to upper junctions of the second rung.

In some examples, the commissure supports are longer than the axial struts.

In some examples, each upper junction of the first rung is circumferentially aligned with a lower junction of the second rung.

In some examples, each angled strut of the first rung, extending between the upper end of the corresponding commissure support and the upper junction of the first rung, has a greater strut length than that of the angled strut of the first rung extending between the same upper junction of the first rung and the upper end of the corresponding axial strut.

In some examples, each cell of the first cell row spans a width of two cells of the second cell row.

In some examples, the second rung comprises four angled struts extending between immediately adjacent axial frame members along the first cell row.

In some examples, the first rung comprises two angled struts extending between immediately adjacent axial frame members along the first cell row.

In some examples, the commissure supports are attached at their upper ends to the upper junctions of the first rung, and are attached at their lower ends to the upper junctions of the second rung.

In some examples, the axial struts are attached at their upper ends to the upper junctions of the first rung, and are attached at their lower ends to the upper junctions of the second rung.

In some examples, the axial struts and the commissure supports have equal lengths.

In some examples, each angled strut of the first rung has a strut length greater than a strut length of any angled strut of the second rung.

In some examples, each angled strut of the first rung has a strut width greater than a strut width of any angled strut of the second rung.

In some examples, the first cell row comprises six cells and the second cell row comprises nine cells.

In some examples, the first cell row comprises six cells and the second cell row comprises twelve cells.

In some examples, a single one of the axial struts is disposed between immediately adjacent commissure supports.

In some examples, a single one of the commissure supports is disposed between immediately adjacent axial struts.

In some examples, the plurality of axial struts comprises three axial struts, and wherein the plurality of commissure supports comprises three commissure supports.

In some examples, the axial struts and the commissure supports are alternately arranged around the circumference of the first cell row.

In some examples, each cell of the first cell row comprises a free apex defined by the second rung.

In some examples, each free apex comprises an arcuate region defined between an upper curved surface and a lower curved surface thereof.

In some examples, a width of the arcuate region of each free apex is less than a width of an angled strut extending therefrom.

In some examples, the cells of the first cell row comprise elongated wide cells and elongated narrow cells, wherein the elongated wide cells are wider, in the circumferential direction, than the elongated narrow cells.

In some examples, each elongated wide cell spans a width of one and a half cells of the second cell row.

In some examples, each elongated narrow cell and each cell of the second cell row have equal widths in the circumferential direction.

In some examples, each elongated wide cell comprises one of the plurality of commissure supports, one of the plurality of axial struts, three angled struts of the first rung extending between the corresponding commissure support and the axial strut, and three angled struts of the second rung extending between the corresponding commissure support and the axial strut.

In some examples, each elongated narrow cell comprises two of the plurality of axial struts, two angled struts of the first rung extending between the corresponding axial struts, and two angled struts of the second rung extending between the corresponding axial struts.

In some examples, two axial struts are disposed between immediately adjacent commissure supports.

In some examples, the elongated wide cells and the elongated narrow cells are alternately arranged around the circumference of the first cell row.

In some examples, each elongated wide cell comprises a free apex defined by the second rung.

In some examples, each free apex comprises an arcuate region defined between an upper curved surface and a lower curved surface thereof.

In some examples, a width of the arcuate region of each free apex is less than a width of an angled strut extending therefrom.

In some examples, each upper junction of the first rung comprises an arcuate region defined between an upper curved surface and a lower curved surface thereof.

In some examples, a width of the arcuate region of each upper junction of the first rung is less than a width of an angled strut extending therefrom.

In some examples, each commissure support further comprises an upper strut portion extending between the commissure window and the upper end of the commissure support.

In some examples, each commissure support further comprises a lower strut portion extending between the commissure window and the lower end of the commissure support.

In some examples, each commissure support further comprises a lower strut portion extending between the commissure window and the lower end of the commissure support.

In some examples, further comprising a fourth rung of angled struts distal to the third rung.

In some examples, further comprising a third cell row comprising a plurality of cells, each cell of the third cell row defined between two angled struts of the third rung and two angled struts of the fourth rung.

In some examples, further comprising a valvular structure mounted inside the frame and comprising a plurality of leaflets, each leaflet comprising a free edge closer to the outflow end of the frame, and a cusp edge along which the leaflet is coupled to the frame.

In some examples, the cusp edge is coupled to two angled struts of the second rung, two angled struts of the third rungs, and two angled struts of the third rung.

In some examples, an apex of the cusp edge is located at the inflow end of the frame.

In some examples, the apex of the cusp edge is not connected to the frame.

In some examples, the apex of the cusp edge is aligned with an inflow apex of the frame.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, and a plurality of axial frame members extending between upper ends and lower ends thereof. The second rung is distal to the first rung. The axial frame members comprise a plurality of commissure supports and a plurality of axial struts. Each commissure support comprises a commissure window and an upper strut portion extending between the commissure window and the upper end. Each commissure support is attached at its upper end to an upper junction of the first rung, and is attached at its lower end to a lower junction of the second rung. Each axial strut is attached at its upper end to a lower junction of the first rung, and is attached at its lower end to an upper junction of the second rung.

In some examples, the first rung comprises three angled struts extending between immediately adjacent axial frame members.

In some examples, the second rung comprises three angled struts extending between immediately adjacent axial frame members.

In some examples, each commissure support further comprises a lower strut portion extending between the commissure window and the lower end of the commissure support.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, and a plurality of axial frame members extending between upper ends and lower ends thereof. The second rung is distal to the first rung. The axial frame members comprise a plurality of commissure supports and a plurality of axial struts. Each commissure support comprises a commissure window and at least one strut portion axially extending from the commissure window. Each commissure support is attached at its upper end to a lower junction of the first rung, and is attached at its lower end to an upper junction of the second rung. Each axial strut is attached at its upper end to an upper junction of the first rung, and is attached at its lower end to a lower junction of the second rung.

In some examples, the first rung comprises three angled struts extending between immediately adjacent axial frame members.

In some examples, the second rung comprises three angled struts extending between immediately adjacent axial frame members.

In some examples, the at least one strut portion comprises an upper strut portion extending between the commissure window and the upper end of the commissure support.

In some examples, the at least one strut portion further comprises a lower strut portion extending between the commissure window and the lower end of the commissure support.

In some examples, the at least one strut portion comprises a lower strut portion extending between the commissure window and the lower end of the commissure support.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, and a plurality of axial frame members extending between upper ends and lower ends thereof. The second rung is distal to the first rung. The axial frame members comprise a plurality of commissure supports and a plurality of axial struts. Each commissure support comprises a commissure window and at least one strut portion axially extending from the commissure window. Each axial strut has a strut length equal to a strut length of any of the commissure supports.

In some examples, the first rung comprises three angled struts extending between immediately adjacent axial frame members.

In some examples, the second rung comprises three angled struts extending between immediately adjacent axial frame members.

In some examples, each commissure support is attached at its upper end to a lower junction of the first rung, and is attached at its lower end to a lower junction of the second rung.

In some examples, each axial strut is attached at its upper end to an upper junction of the first rung, and is attached at its lower end to an upper junction of the second rung.

In some examples, each commissure support is attached at its upper end to an upper junction of the first rung, and is attached at its lower end to an upper junction of the second rung.

In some examples, each axial strut is attached at its upper end to a lower junction of the first rung, and is attached at its lower end to a lower junction of the second rung.

In some examples, the at least one strut portion comprises an upper strut portion extending between the commissure window and the upper end of the commissure support.

In some examples, the at least one strut portion further comprises a lower strut portion extending between the commissure window and the lower end of the commissure support.

In some examples, the at least one strut portion comprises a lower strut portion extending between the commissure window and the lower end of the commissure support.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, and a plurality of axial frame members extending between upper ends and lower ends thereof. The second rung is distal to the first rung. Each axial frame member connected at an upper end thereof to the first rung, and at a lower end thereof to the second rung.

In some examples, the first rung comprises two angled struts extending between immediately adjacent axial frame members.

In some examples, the second rung comprises more than two angled struts extending between immediately adjacent axial frame members.

In some examples, the axial frame members comprise a plurality of axial struts and a plurality of commissure supports, each commissure support comprising a commissure window.

In some examples, the second rung comprises three angled struts extending between immediately adjacent axial frame members.

In some examples, each commissure support is attached at its upper end to a lower junction of the first rung, and is attached at its lower end to a lower junction of the second rung.

In some examples, each axial strut is attached at its upper end to a lower junction of the first rung, and is attached at its lower end to an upper junction of the second rung.

In some examples, each upper junction of the first rung comprises an arcuate region defined between an upper curved surface and a lower curved surface thereof.

In some examples, a width of the arcuate region of each upper junction of the first rung is less than a width of an angled strut extending therefrom.

In some examples, each upper junction of the first rung is circumferentially aligned with a lower junction of the second rung.

In some examples, each angled strut of the first rung, extending between the upper end of the corresponding commissure support and the upper junction of the first rung, has a greater strut length than that of the angled strut of the first rung extending between the same upper junction of the first rung and the upper end of the corresponding axial strut.

In some examples, the second rung comprises four angled struts extending between immediately adjacent axial frame members.

In some examples, each commissure support is attached at its upper end to an upper junction of the first rung, and is attached at its lower end to an upper junction of the second rung.

In some examples, each axial strut is attached at its upper end to an upper junction of the first rung, and is attached at its lower end to an upper junction of the second rung.

In some examples, each upper junction of the first rung comprises an arcuate region defining an upper curved surface opposite to the axial frame member attached to the upper junction of the first rung.

In some examples, each commissure support further comprises an upper strut portion extending between the commissure window and the upper end of the commissure support.

In some examples, each commissure support further comprises a lower strut portion extending between the commissure window and the lower end of the commissure support.

In some examples, each commissure support further comprises a lower strut portion extending between the commissure window and the lower end of the commissure support.

In some examples, further comprising free apices defined by the second rung between immediately adjacent axial frame members.

In some examples, each free apex comprises an arcuate region defined between an upper curved surface and a lower curved surface thereof.

In some examples, wherein a width of the arcuate region of each free apex is less than a width of an angled strut extending therefrom.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, and a first cell row. The second rung is distal to the first rung. The first cell row comprises a plurality of commissure supports, and a plurality of axial struts. Each commissure support is attached at its upper end to a lower junction of the first rung, and is attached at its lower end to a lower junction of the second rung. Each axial strut is attached at its upper end to an upper junction of the first rung, and is attached at its lower end to an upper junction of the second rung.

The first cell row further comprises a plurality of elongated wide cells, and a plurality of elongated narrow cells. Each elongated wide cell comprises one of the plurality of commissure supports, one of the plurality of axial struts, three angled struts of the first rung extending between the corresponding commissure support and the axial strut, and three angled struts of the second rung extending between the corresponding commissure support and the axial strut. Each elongated narrow cell comprises two of the plurality of axial struts, two angled struts of the first rung extending between the corresponding axial struts, and two angled struts of the second rung extending between the corresponding axial struts.

In some examples, two axial struts are disposed between immediately adjacent commissure supports.

In some examples, the elongated wide cells and the elongated narrow cells are alternately arranged around the circumference of the first cell row.

In some examples, each elongated wide cell comprises a free apex defined by the second rung.

In some examples, a width of the arcuate region of each free apex is less than a width of an angled strut extending therefrom.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, a third rung of angled struts interconnected at upper junctions and lower junctions of the third rung, and a fourth rung of angled struts interconnected at upper junctions and lower junctions of the fourth rung. The second rung is distal to the first rung. The third rung is distal to the second rung. The fourth rung is distal to the third rung. The frame further comprises a first cell row comprising a plurality of upper cells, and a second cell row. Each upper cell is defined between two angled struts of the first rung and four angles struts of the second rung.

The second cell row comprises a plurality of commissure supports, a plurality of axial struts, a plurality of elongated wide cells, and a plurality of elongated narrow cells. Each commissure support is attached at its upper end to an upper junction of the second rung, and is attached at its lower end to an upper junction of the third rung. Each axial strut is attached at its upper end to a lower junction of the second rung, and is attached at its lower end to an upper junction of the fourth rung. Each elongated wide cell comprises one of the plurality of commissure supports, one of the plurality of axial struts, three angled struts of the second rung extending between the corresponding commissure support and the axial strut, and three angled struts of the third rung extending between the corresponding commissure support and the axial strut. Each elongated narrow cell comprises two of the plurality of axial struts, two angled struts of the second rung extending between the corresponding axial struts, and two angled struts of the fourth rung extending between the corresponding axial struts.

In some examples, the third rung is discontinuous in the circumferential direction, being devoid of angled struts at regions of the elongated narrow cells.

In some examples, each upper cell is wider, in the circumferential direction, than any of the elongated wide cells and the elongated narrow cells.

In some examples, the frame further comprises a third cell row comprising a plurality of cells, each cell of the third cell row comprising two angled struts of the third rung and two angled struts of the fourth rung.

In some examples, the third cell row is discontinuous in the circumferential direction, being devoid of cells at regions of the elongated narrow cells.

In some examples, each upper cell spans a width of two cells of the third cell row.

In some examples, two axial struts are disposed between immediately adjacent commissure supports.

In some examples, the elongated wide cells and the elongated narrow cells are alternately arranged around the circumference of the second cell row.

In some examples, each elongated wide cell comprises a free apex defined by the second rung, and a free apex defined by the third rung.

In some examples, each free apex of the second rung and the third rung comprises an arcuate region defined between an upper curved surface and a lower curved surface thereof.

In some examples, wherein a width of the arcuate region of each free apex is less than a width of the angled strut extending therefrom.

In some examples, a strut length of each angled strut of the first rung is greater than a strut length of any of the angled struts of the second rung, the third rung, and the fourth rung.

In some examples, all angled struts of the second rung, the third rung, and the fourth rung, have equal strut lengths.

In some examples, each angled strut of the first rung has a strut width greater than a strut width of any angled strut of the second rung, the third rung, and the fourth rung.

In some examples, the upper junctions of the first rung are outflow apices of the frame, and wherein each outflow apex comprises an arcuate region defined between an upper curved surface and a lower curved surface thereof.

In some examples, a width of the arcuate region of each outflow apex is less than a width of the angled strut extending therefrom.

In some examples, wherein the frame further comprises a fifth rung comprising a plurality of angled struts, and a fourth cell row comprising a plurality of cells, each cell of the fourth cell row comprising two angled struts of the fourth rung and two angled struts of the fifth rung.

In some examples, the frame further comprises a sixth rung comprising a plurality of angled struts, and a fifth cell row comprising a plurality of cells, each cell of the fifth cell row comprising two angled struts of the fifth rung and two angled struts of the sixth rung.

In some examples, further comprising a valvular structure mounted inside the frame and comprising a plurality of leaflets, each leaflet comprising a free edge closer to the first rung of the frame, and a cusp edge along which the leaflet is coupled to the frame.

In some examples, an apex of the cusp edge is located at an inflow end of the frame.

In some examples, the apex of the cusp edge is not connected to the frame.

In some examples, the apex of the cusp edge is aligned with an inflow apex of the frame.

In some examples, the plurality of leaflets comprises three leaflets.

According to some aspects of the disclosure, there is provided a prosthetic valve comprising a frame movable between a radially compressed state and a radially expanded state. The frame comprises a first rung of angled struts interconnected at upper junctions and lower junctions of the first rung, a second rung of angled struts interconnected at upper junctions and lower junctions of the second rung, a third rung of angled struts interconnected at upper junctions and lower junctions of the third rung, and a fourth rung of angled struts interconnected at upper junctions and lower junctions of the fourth rung. The second rung is distal to the first rung. The third rung is distal to the second rung. The fourth rung is distal to the third rung.

The frame further comprises a first cell row, a second cell row, and a third cell row, each of the cell rows comprising a plurality of cells. The first cell row further comprises a plurality of axial frame members. Each axial frame member connected at an upper end thereof to a lower junction of the first rung, and at a lower end thereof to a lower junctions of the second rung.

In some examples, each cell of the first cell row comprises two immediately adjacent axial frame members, two elongated struts of the first rung extending between the two immediately adjacent axial frame members; and two elongated struts of the second rung extending between the two immediately adjacent axial frame members.

In some examples, each cell of the second cell row defined between two angled struts of the second rung and four angled struts of the third rung.

In some examples, each cell of the third cell row defined between two angled struts of the third rung and two angled struts of the fourth rung.

In some examples, each cell of the first cell row spans a width, in the circumferential direction, of two cells of the third cell row.

In some examples, each cell of the first cell row spans a width, in the circumferential direction, of two cells of the third cell row.

In some examples, the upper junctions of the first rung are outflow apices of the frame, and wherein each outflow apex comprises an arcuate region defined between an upper curved surface and a lower curved surface thereof.

In some examples, a width of the arcuate region of each outflow apex is less than a width of the angled strut extending therefrom.

In some examples, each cell of the first cell row further comprises a free apex defined by the second rung.

In some examples, each free apex comprises an arcuate region defined between an upper curved surface and a lower curved surface thereof.

In some examples, a width of the arcuate region of each free apex is less than a width of an angled strut extending therefrom.

In some examples, a strut length of each angled strut of the first rung and the second rung is greater than a strut length of any of the angled struts of the third rung and the fourth rung.

In some examples, all angled struts of the first rung and the second rung have equal strut lengths.

In some examples, all angled struts of the third rung and the fourth rung have equal strut lengths.

In some examples, each angled strut of the first rung has a strut width greater than a strut width of any angled strut of the third rung and the fourth rung.

In some examples, each angled strut of the second rung has a strut width greater than a strut width of any angled strut of the third rung and the fourth rung.

In some examples, the axial frame members comprise a plurality of axial struts and a plurality of commissure supports.

In some examples, the commissure support is configured to support a commissure of a valvular structure that can be mounted in the frame.

In some examples, each of the plurality of commissure supports comprises a commissure window.

In some examples, the commissure window comprises a window opening between two window struts of the commissure window.

In some examples, each commissure support further comprises an upper strut portion extending between the commissure window and the upper end of the commissure support.

In some examples, each commissure support further comprises a lower strut portion extending between the commissure window and the lower end of the commissure support.

In some examples, each commissure support further comprises a lower strut portion extending between the commissure window and the lower end of the commissure support.

In some examples, a single one of the axial struts is disposed between immediately adjacent commissure supports.

In some examples, a single one of the commissure supports is disposed between immediately adjacent axial struts.

In some examples, the plurality of axial struts comprises three axial struts, and wherein the plurality of commissure supports comprises three commissure supports.

In some examples, the axial struts and the commissure supports are alternately arranged around the circumference of the first cell row.

In some examples, the frame further comprises a fifth rung comprising a plurality of angled struts, and a fourth cell row comprising a plurality of cells, each cell of the fourth cell row comprising two angled struts of the fourth rung and two angled struts of the fifth rung.

In some examples, the frame further comprises a sixth rung comprising a plurality of angled struts, and a fifth cell row comprising a plurality of cells, each cell of the fifth cell row comprising two angled struts of the fifth rung and two angled struts of the sixth rung.

In some examples, further comprising a valvular structure mounted inside the frame and comprising a plurality of leaflets, each leaflet comprising a free edge closer to the first rung of the frame, and a cusp edge along which the leaflet is coupled to the frame.

In some examples, an apex of the cusp edge is located at an inflow end of the frame.

In some examples, the apex of the cusp edge is not connected to the frame.

In some examples, the apex of the cusp edge is aligned with an inflow apex of the frame.

In some examples, the plurality of leaflets comprises three leaflets.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures:

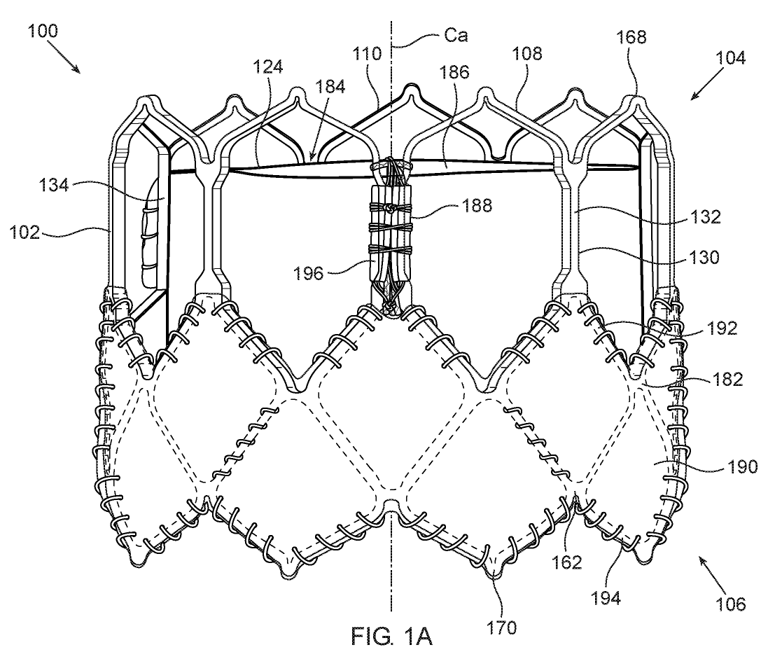

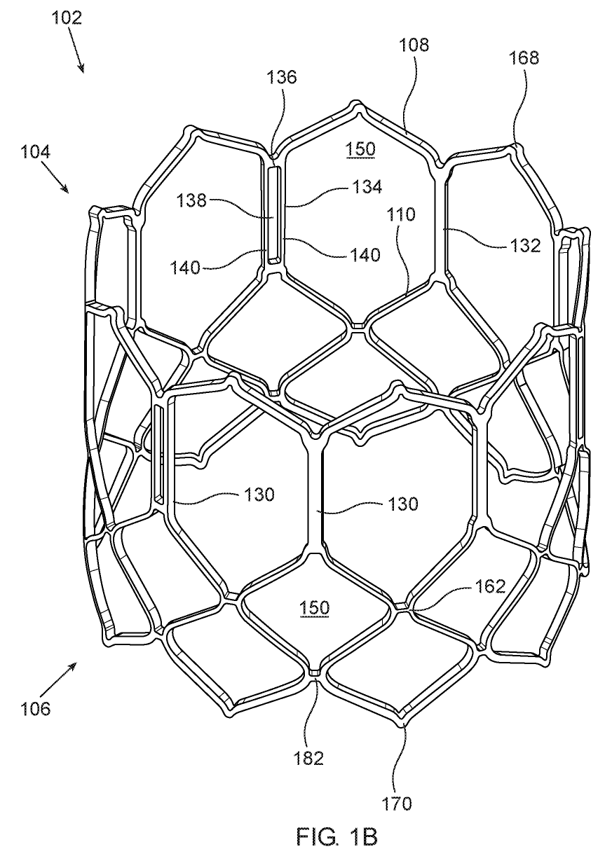

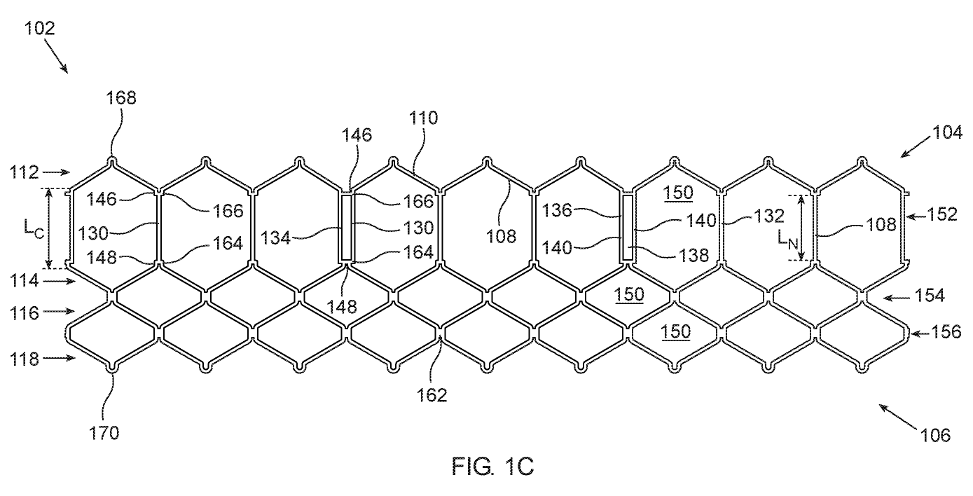

FIG. 1A is a perspective side view of an exemplary prosthetic valve.

FIG. 1B is a perspective view of an annular frame of the prosthetic valve of FIG. 1A.

FIG. 1C is a flattened view of the frame of FIG. 1B.

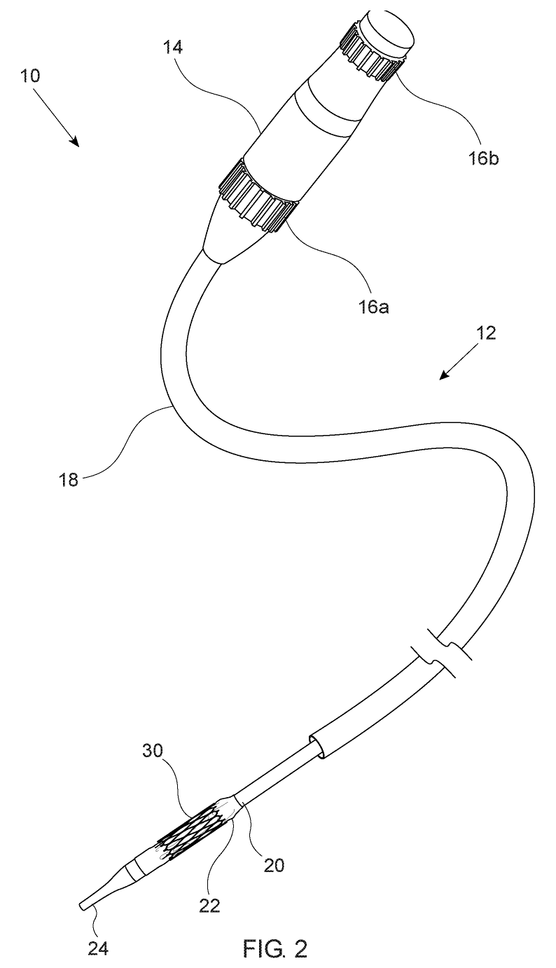

FIG. 2 shows an exemplary delivery apparatus carrying an exemplary prosthetic valve.

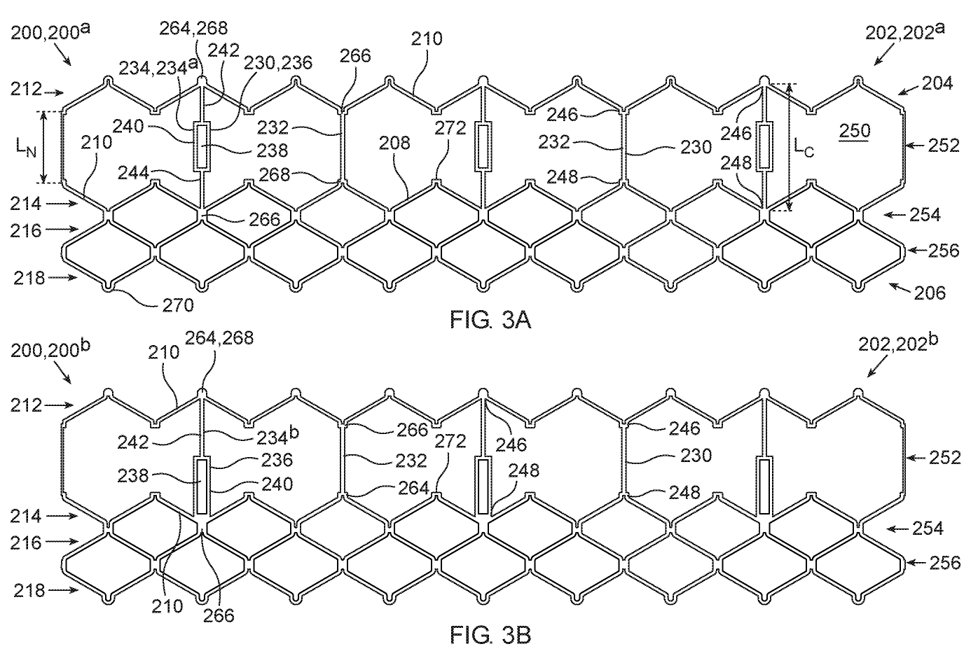

FIGS. 3A-3B show exemplary frames that include commissure supports which are longer than the axial struts.

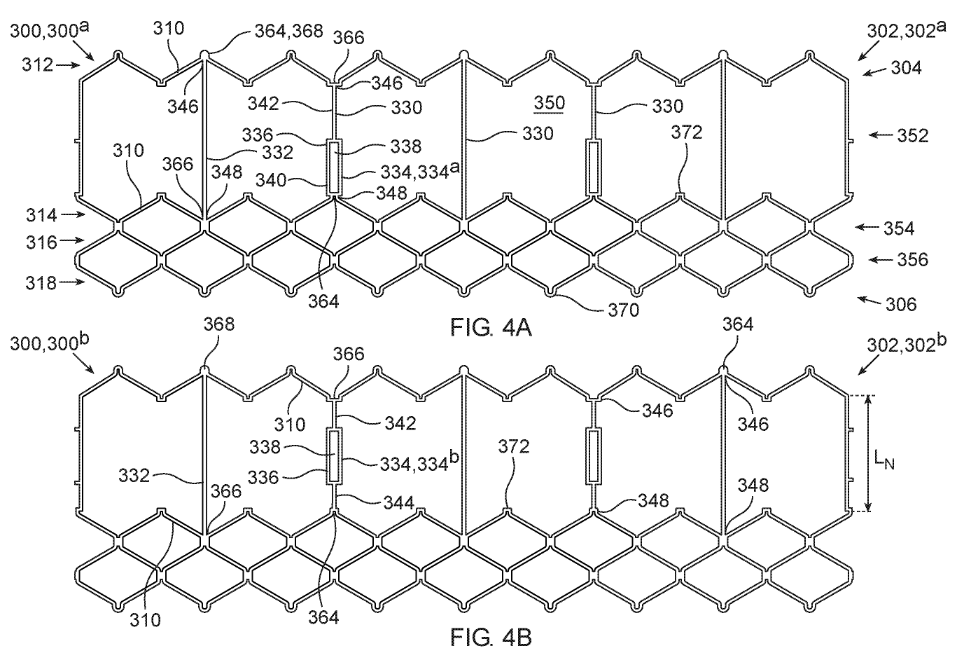

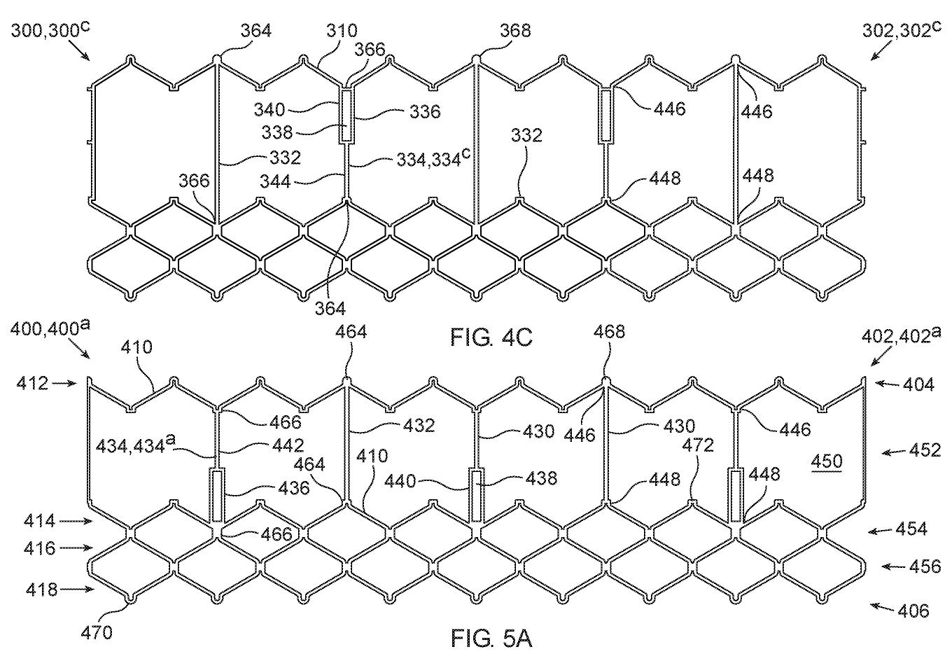

FIGS. 4A-4C show exemplary frames that include axial struts which are longer than the commissure supports.

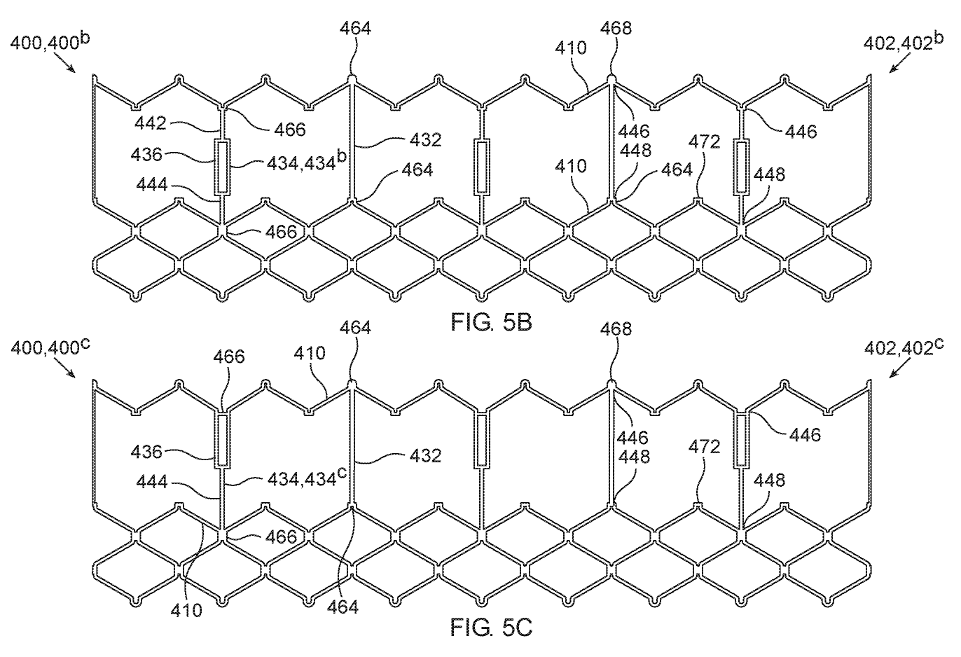

FIGS. 5A-5C show exemplary frames that include commissure supports which are distally offset relative to the axial struts.

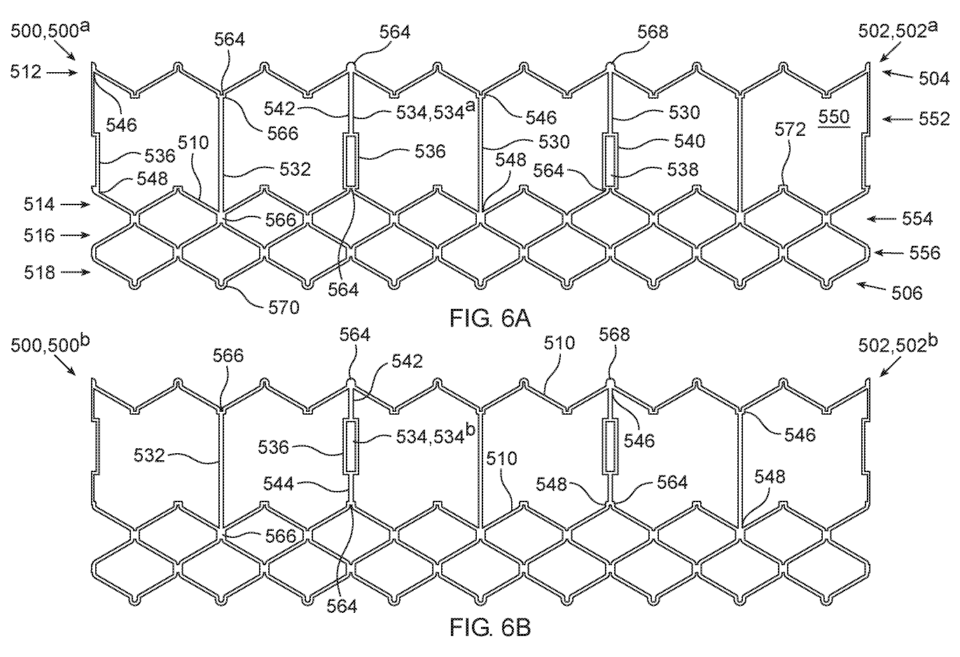

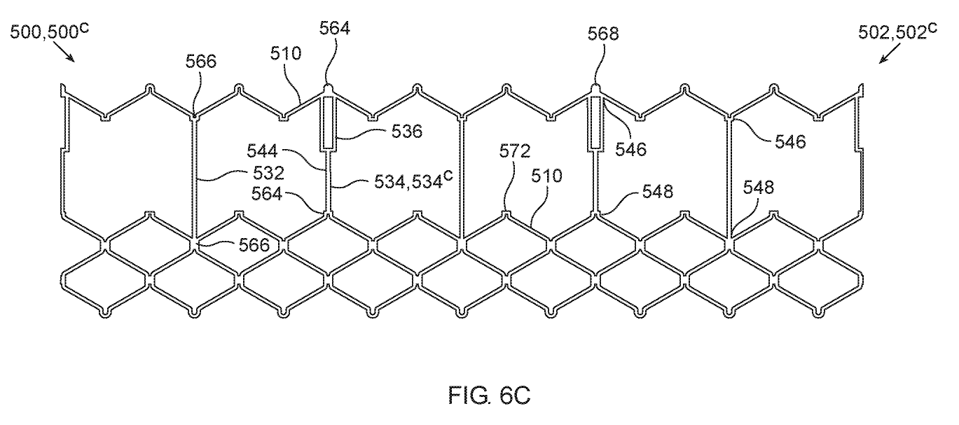

FIGS. 6A-6C show exemplary frames that include axial struts which are distally offset relative to the commissure supports.

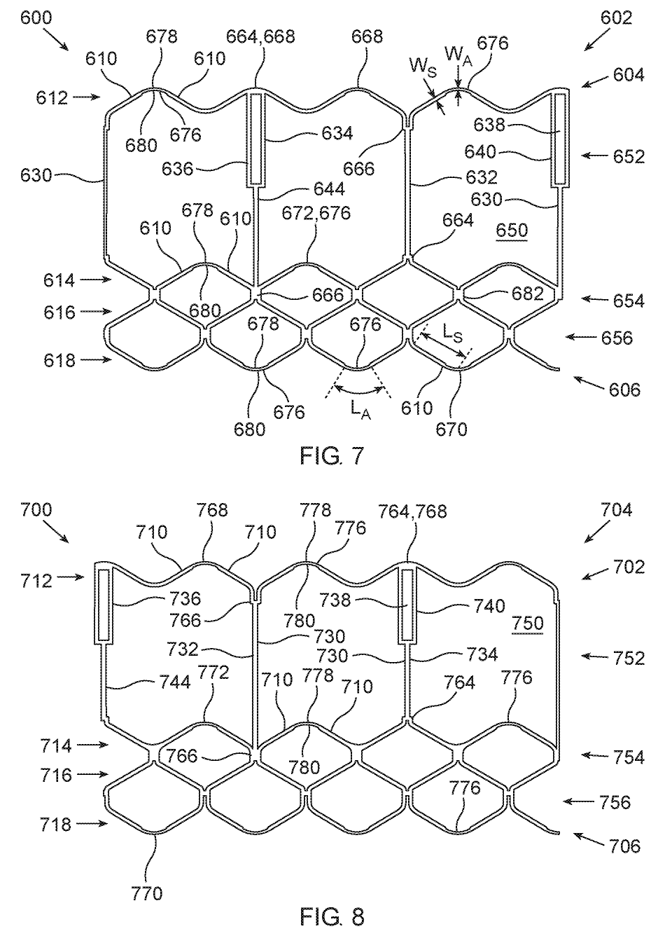

FIG. 7 shows a portion of an exemplary frame that includes commissure supports which are longer than the axial struts, and defining apices that include arcuate regions.

FIG. 8 shows a portion of an exemplary frame that includes commissure supports and axial struts having equal lengths, and defining apices that include arcuate regions.

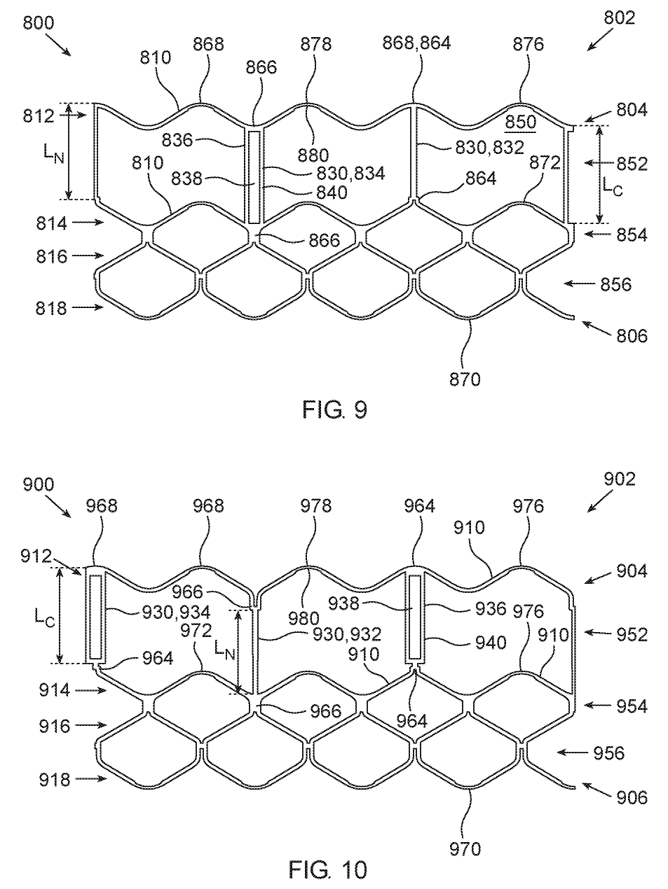

FIG. 9 shows a portion of an exemplary frame that includes commissure supports which are distally offset relative to the axial struts, and defining apices that include arcuate regions.

FIG. 10 shows a portion of an exemplary frame that includes commissure supports which are proximally offset relative to the axial struts, and defining apices that include arcuate regions.

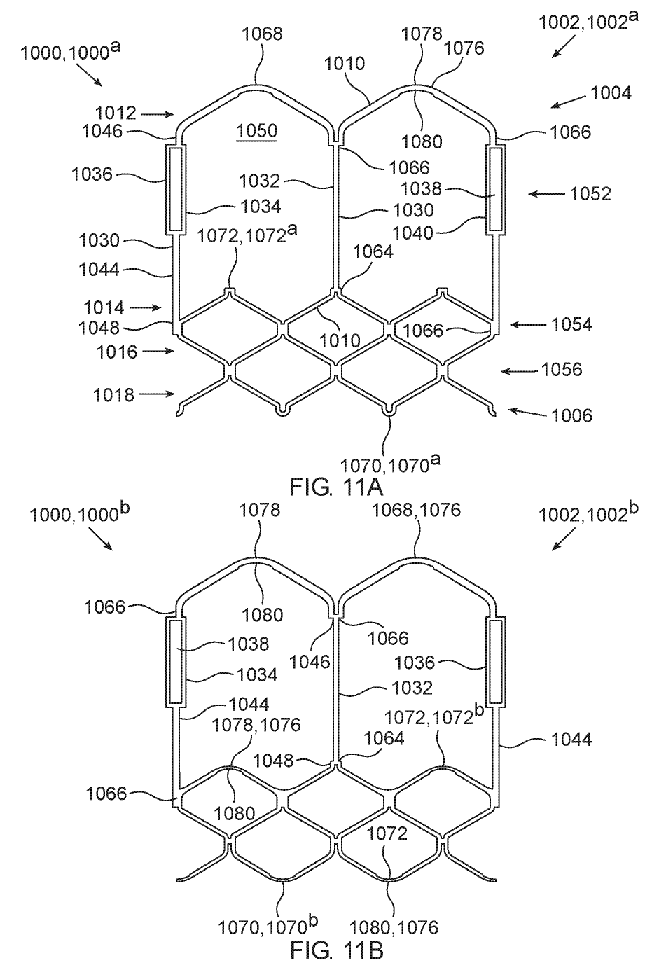

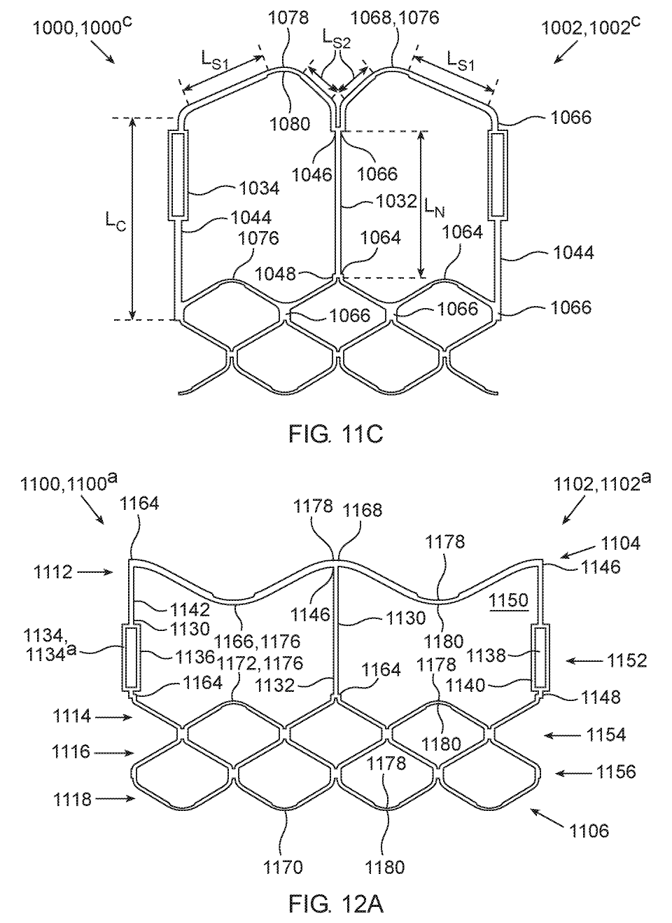

FIGS. 11A-11C show exemplary frames that include two angled struts along the first rung, and three angled struts along the second rung, extending between immediately adjacent axial frame members.

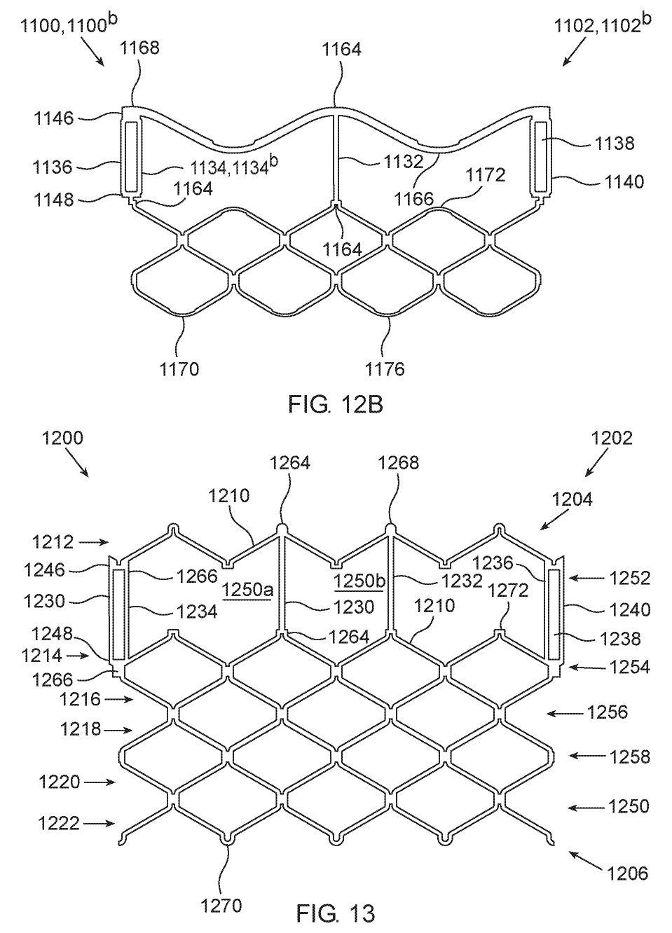

FIGS. 12A-12B show exemplary frames that include two angled struts along the first rung, and four angled struts along the second rung, extending between immediately adjacent axial frame members.

FIG. 13 shows a portion of an exemplary frame including two axial struts disposed between immediately adjacent commissure supports.

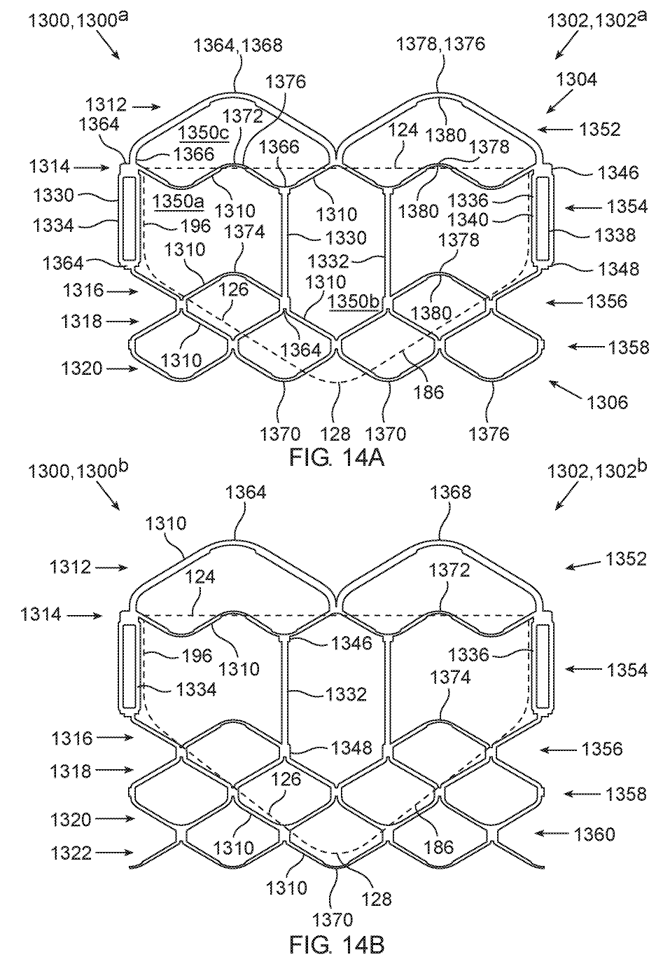

FIGS. 14A-14B show exemplary frames that include an first cell row with upper cells, and a second cell row with elongated wide cells and elongated narrow cells.

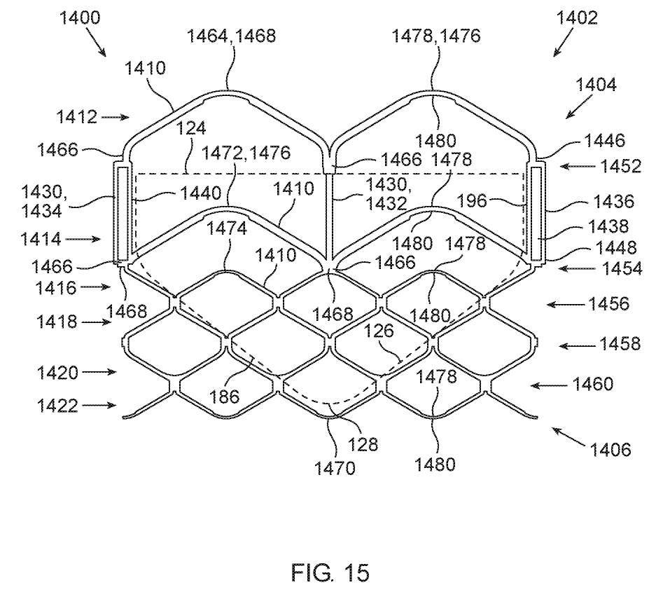

FIG. 15 shows a portion of an exemplary frame wherein each cell of the first and second cell rows spans a width, in the circumferential direction, of two cells of subsequent cell rows.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms "have" or “includes” means “comprises”. Further, the terms “coupled”, “connected”, and "attached", as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient’s body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

FIGS. 1A and 1B show perspective views of an example of a prosthetic valve 100, with and without soft components (such as skirts and a leaflet assembly), respectively. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus 12 (see FIG. 2) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve 100 of the current disclosure may include any prosthetic valve configured to be mounted within the native aortic valve, the native mitral valve, the native pulmonary valve, and the native tricuspid valve.

It is to be understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus 12 (see FIG. 2). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Patent No. 10,603,165, International Application No. PCT/US2021/052745, and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term "plurality", as used herein, means more than one.

The prosthetic valve 100 comprises an inflow end 106, an outflow end 104, and a central longitudinal axis Ca extending in a direction from the inflow end 106 to the outflow end 104. In some instances, the inflow end 106 is the distal end of the prosthetic valve 100, and the outflow end 104 is the proximal end of the prosthetic valve 100. Alternatively, depending for example on the delivery approach of the valve, the inflow end can be the proximal end of the prosthetic valve, and the outflow end can be the distal end of the prosthetic valve.

The term “proximal”, as used herein, generally refers to a position, direction, or portion of a device or a component of a device, which is closer to the user (for example, during an implantation procedure) and further away from the implantation site.

The term “distal”, as used herein, generally refers to a position, direction, or portion of a device or a component of a device, which is further away from the user and closer to the implantation site.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

The valve 100 comprises an annular frame 102 movable between a radially compressed state and a radially expanded state, and a valvular structure 184 mounted within the frame 102. FIG. 1C shows the frame 102 in a flat configuration for purposes of illustration. The frame 102 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter (not shown), and then expanded inside a patient by an inflatable balloon or equivalent expansion mechanism. Alternatively or additionally, the frame 102 can be made of shape-memory materials such as, but not limited to, nickel titanium alloy (e.g., Nitinol).

In the example illustrated in FIGS. 1A-1B, the frame 102 is an annular, stent-like structure comprising a plurality of intersecting struts 108. In this application, the term "strut" 108 encompasses vertical struts, angled or curved struts, support posts, commissure windows, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define a plurality of cells 150 arranged in several cell rows. The frame 102 can have a cylindrical or substantially cylindrical shape having a constant diameter from the inflow end 106 to the outflow end 104 as shown, or the frame can vary in diameter along the height of the frame, as disclosed in US Pat. No. 9,155,619, which is incorporated herein by reference.

Struts 108 comprise angled struts 110 and axial frame members 130. The term "axial frame member" refers to a strut or a component of the frame 102 that generally extends in an axial direction, while the term "angled strut" generally refers to a strut that can extend at an angle relative to an axial line intersecting therewith along a plane defined by the frame 102. It is to be understood that the term "angled strut" encompasses both linear angled struts and curved struts.

FIGS. 1A-1B show an exemplary prosthetic valve 100 that can be representative of, but is not limited to, a balloon expandable prosthetic valve. The frame 102 of the prosthetic valve 100 illustrated in FIGS. 1B-1C comprises three cell rows, namely a first cell row 152, a second cell row 154, a third cell row 156, wherein the first cell row 152 is closer to the outflow end 104 than the second and third cell rows, and the third cell row 156 is closer to the inflow end 106 than the first and second cell rows. The three rows of cells are shown by way of illustration and not limitation, and it is to be understood that more or less cell rows may be defined by other frame configurations.

Two or more struts 108 can intersect at junctions 162, which can be equally or unequally spaced apart from each other. The struts 108 may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

The valvular structure 184 comprises a plurality of leaflets 186 (e.g., three leaflets), positioned at least partially within the frame 102, and configured to regulate flow of blood through the prosthetic valve 100 from the inflow end 106 to the outflow end 104. While three leaflets 186 arranged to collapse in a tricuspid arrangement, are shown in the example illustrated in FIG. 1A, it will be clear that a prosthetic valve 100 can include any other number of leaflets 186. Adjacent leaflets 186 can be arranged together to form commissures 188 that are coupled (directly or indirectly) to respective portions of the frame 102, thereby securing at least a portion of the valvular structure 184 to the frame 102. In some examples, each leaflet 186 can comprise opposing tabs 196. Each tab 196 can be secured to an adjacent tab 196 of an adjacent leaflet 186 to form a commissure 188 that is secured to the frame 102.

The leaflets 186 can be made from, in whole or part, biological material (e.g., pericardium), bio-compatible synthetic materials, or other such materials. Further details regarding transcatheter prosthetic heart valves, including the manner in which the valvular structure 184 can be coupled to the frame 102 of the prosthetic valve 100, can be found, for example, in U.S. Patent Nos. 6,730,118, 7,393,360, 7,510,575, 7,993,394, 8,652,202, and 11,135,056, all of which are incorporated herein by reference in their entireties.

The prosthetic valve 100 can further comprise at least one skirt or sealing member. In some examples, a prosthetic valve can include an inner skirt (not shown) secured to an inner surface of the frame 102, and configured to function, for example, as a sealing member to prevent or decrease perivalvular leakage. An inner skirt can further function as an anchoring region for the leaflets 186 to the frame 102, and/or function to protect the leaflets 186 against damage which may be caused by contact with the frame 102, for example during valve crimping or during working cycles of the prosthetic valve 100. In some examples, a prosthetic valve 100 can include an outer skirt 190 mounted on an outer surface of the frame 102, and configure to function, for example, as a sealing member retained between the frame 102 and the surrounding tissue of the native annulus against which the prosthetic valve is mounted, thereby reducing risk of paravalvular leakage (PVL) past the prosthetic valve 100.

Any of an inner skirt and/or outer skirt 190 can be made of various suitable biocompatible materials, such as, but not limited to, various synthetic materials (e.g., PET) or natural tissue (e.g. pericardial tissue). In some examples, an inner skirt comprises a single sheet of material that extends continuously around the inner surface of the frame 102. In some examples, the outer skirt 190 comprises a single sheet of material that extends continuously around the outer surface of the frame 102. The outer skirt 190 can extend between an inflow end 194, which is closer to the inflow end 106 of the frame 102, and an outflow end 192 which is closer to the outflow end 104 of the frame.

FIGS. 1B-1C show the frame 102 of the prosthetic valve 100 with the other components, such as leaflets and skirts, removed. While FIG. 1B shows the frame 102 in an annular configuration, corresponding to its functional configuration. FIG. 1C shows the frame 102 in a flat configuration for purposes of illustration. In the example illustrated in FIGS. 1B-1C, the frame 102 comprises three cell rows 152, 154, 156, each row comprising a plurality of cells 150 extending circumferentially such that each cell 150 is directly coupled to two circumferentially adjacent cells 150 on both sides thereof within the same row of cells. The term "cell", as used herein, refers to a closed cell, having an enclosed perimeter defined by at least four struts 108.

The junctions 162 include outflow apices 168 and inflow apices 170 which are the uppermost and lowermost junctions, respectively, of the frame 102, and intermediate junctions 182 disposed between the inflow end 106 and outflow end 104.

In some examples, cells 150 are coupled to adjacent cells 150 within the same cell row, such as within a first cell row 152, via axial frame members 130. Axial frame members 130 can include, in some examples, axially extending commissure supports 134 and non-commissural axial struts 132. An axially extending commissure support 134 can include, in some examples, a commissure window 136 defining a window opening 138 between two window struts 140, wherein the commissure window 136 is configured to support a corresponding commissure 188 of the valvular structure 184. The axial frame members 130, including non-commissural axial struts 132 and axially extending commissure supports 134, can be parallel to each other and/or to the central longitudinal axis Ca of the prosthetic valve 100.

The terms "axially-extending commissure support" and "commissure support", as used herein, are interchangeable, and refer to an axial frame member configured to support a commissure 188 attachable or attached thereto, and may optionally include a commissure window 136, though in some examples, a commissure support 134 can be configured to mount a commissure 188 in any other suitable manner, such as by supporting portions of the valvular structure 184 that can be wrapped therearound, can include apertures through which sutures for attached the commissures can be passed, and the like. The terms "non-commissural axial strut" and "axial strut", as used herein, are interchangeable, and refer to a an axial frame member configured to remain unattached to the valvular structure 184. That is to say, an axial struts 132 is not configured to mount a commissure, and may be devoid of a commissure window.

In the example illustrated in FIGS. 1B-1C, the frame 102 comprises four rungs of angled struts 110, including a first rung 112 of angled struts that can be closer to the outflow end 104 relative to other rungs of struts, a second rung 114 of angled struts which is distal to the first rung 112, a third rung 116 of angled struts which is distal to the second rung 114, and a fourth rung 118 of angled struts which is distal to the third rung 116.

The cells 150 of the first cell row 152 of the exemplary valve 100 shown in FIGS. 1B-1C can be generally hexagonal, each cell defined between two angled struts 110 of the first rung 112, two angled struts 110 of the second rung 114, and two axial frame members 130 extending between the first rung 112 and the second rung 114. Cells of the second cell row 154 and the third cell row 156 can be generally diamond-shaped cells, with the cells 150 of the second cell row 154 defined by two angled struts 110 of the second rung 114 and two angled struts 110 of the third rung 116, and cells 150 of the third cell row 156 defined by two angled struts 110 of the third rung 116 and two angled struts 110 of the fourth rung 118.

Each rung of angled struts 110 is circumferentially arranged in a zig-zagged pattern such that the angled struts 110 of each rung are interconnected at upper junctions 164 and at lower junctions 166, wherein the upper junctions 164 are closer to the outflow end 104 while the lower junctions 166 are closer to the inflow end 106. The upper junctions 164 of the first rung 112 can be the outflow apices 168, and the lower junctions 166 of the fourth rung 118 of the exemplary frame 102 illustrated in FIGS. 1B-1C can be the inflow apices 170.

Each axial frame member 130 can vertically extend between an upper end 146 at which the axial frame member 130 is linked to a junction 162 of the first rung 112, and a lower end 148 at which the axial frame member 130 is linked to a junction 162 the second rung 114. In the example illustrated in FIGS. 1B-1C, axial frame members 130 of the first cell row 152 are attached at their upper ends 146 to lower junctions 166 of the first rung 112 of angled struts, and are attached at their lower ends 148 to upper junctions 164 of the second rung 114 of angled struts.

In the example illustrated in FIGS. 1A-1C, the lengths of all axial frame member 130, defined between their respective lower ends 148 and upper ends 146, can be equal. Stated otherwise, the length L_{N} (indicated in FIG. 1C) of a non-commissural axial struts 132 is substantially equal to the length L_{C} of an axially extending commissure support 134 for exemplary frame 102.

At least two angled struts 110 of at least one rung of struts interconnecting the axial frame members 130 can extend between each two immediately adjacent axial frame member 130. The term "immediately adjacent axial frame members" refers to circumferentially adjacent axial frame members 130 that do not include another axial frame member 130 disposed therebetween. In the example illustrated in FIGS. 1A-1C, two angled struts 110 of the first rung 112 extend between each two immediately adjacent axial frame members 130, such as between upper ends 146 thereof, and two angled struts 110 of the second rung 114 similarly extend between each two immediately adjacent axial frame members 130, such as between lower ends 148 thereof. Each cell 150 of the first cell row 152 can have a width (in a circumferential directions, such as between two immediately adjacent axial frame members 130 thereof) that is equal to that of cells 150 in subsequent cell rows, such as cells of the second cell row 154 and third cell row 156.

In some examples, at least one axial strut 132 is disposed between each two immediately adjacent commissure supports 134. The term "immediately adjacent commissure supports" refers to circumferentially adjacent commissure supports 134 that do not include another commissure support 134 disposed therebetween, while one or more axial struts 132 can be disposed at equal or non-equal distances between the two commissure supports 134. In the example illustrated in FIGS. 1A-1C, three axial struts 132 are disposed between each two immediately adjacent commissure supports 134.

FIG. 2 shows a perspective view of an exemplary delivery assembly 10 that includes a delivery apparatus 12 adapted to deliver a prosthetic device, which can be a prosthetic valve 30 described herein. Prosthetic valve 30 carried by delivery apparatus 12 can be any of the exemplary valve disclosed herein, including prosthetic valve 100 described above with respect to FIGS. 1A-1C, or any of prosthetic valve 200, 300, 400, 500, 600, 700, 800, 900, 1000, 1100, 1200, 1300, or 1400, described below with respect to FIGS. 3-15. The delivery apparatus 12 can include a handle 14 and at least one catheter extending therefrom, configured to carry a prosthetic valve 30 in a crimped state through the patient's vasculature. An exemplary delivery assembly 10 comprises an exemplary delivery apparatus 12 configured to carry a balloon expandable prosthetic valve. The delivery apparatus 12 can comprise a balloon catheter 20 having an inflatable balloon 22 mounted on its distal end. A prosthetic device, such as prosthetic valve 30, can be carried in a crimped state over the balloon catheter 20.

In some examples, a delivery apparatus 12 further comprises an outer shaft 18. Optionally, an outer shaft 18 of a delivery apparatus 12 can concentrically extend over the balloon catheter 20.

The outer shaft 18 and the balloon catheter 20 can be configured to be axially movable relative to each other. For example, a proximally oriented movement of the outer shaft 18 relative to the balloon catheter 20, or a distally oriented movement of the balloon catheter 20 relative to the outer shaft 18, can expose the prosthetic valve 30 from the outer shaft 18.

A delivery apparatus 12 can further include a nosecone 24 to facilitate advancement of the delivery apparatus 12 through the patient's vasculature to the site of treatment. A nosecone shaft (concealed from view in FIG. 2) can extend proximally from the nosecone 24 through a lumen of the balloon catheter 20, towards the handle 14.

In FIG. 2, a prosthetic valve 30 is mounted on the balloon 22 and is shown in a crimped state, providing prosthetic valve 30 with a reduced diameter for delivery to the heart via the patient's vasculature. While the prosthetic valve 30 is shown in FIG. 2 as being crimped or mounted on the balloon 22 for delivery to the treatment location, it should be understood that the prosthetic valve can be crimped or mounted at a location different from the location of balloon 22 (e.g., proximal to the balloon 22) and repositioned over the balloon at some time before inflating the balloon and deploying the prosthetic valve. This off-balloon delivery allows the prosthetic valve to be crimped to a lower profile than would be possible if the prosthetic valve was crimped on top of the balloon 22. The lower profile permits the clinician to more easily navigate the delivery apparatus (including the crimped prosthetic valve) through a patient's vasculature to the treatment location. The lower profile of the crimped prosthetic valve can be particularly helpful when navigating through portions of the patient's vasculature which are particularly narrow, such as the iliac artery.

The proximal ends of the balloon catheter 20, the outer shaft 18, and/or the nosecone shaft, can be coupled to the handle 14. During delivery, the handle 14 can be maneuvered by an operator (e.g., a clinician or a surgeon) to axially advance or retract components of the delivery apparatus 12, such as the nosecone shaft, the outer shaft 18, and/or the balloon catheter 20, through the patient's vasculature and/or along the target site of implantation, as well as to inflate the balloon 22 mounted on the balloon catheter 20, for example to expand a prosthetic valve 30 mounted on the balloon 22, and to deflate the balloon 22 and retract the delivery apparatus 12, for example once the prosthetic valve 30 is mounted in the implantation site.

The handle 14 can include a steering mechanism configured to adjust the curvature of a distal end portion of the delivery apparatus 12. In the illustrated example, the handle 14 includes an adjustment member, such as the illustrated rotatable knob 16a, which in turn is operatively coupled to the proximal end portion of a pull wire (not shown). The pull wire can extend distally from the handle 14 through the outer shaft 18 and has a distal end portion affixed to the outer shaft 18 at or near the distal end of the outer shaft 18. Rotating the knob 16a can increase or decrease the tension in the pull wire, thereby adjusting the curvature of the distal end portion of the delivery apparatus 12. Further details on steering or flex mechanisms for the delivery apparatus can be found in U.S. Pat. No. 9,339,384, which is incorporated by reference herein.

In some examples, the handle 14 can include an adjustment members such as the illustrated rotatable knob 16b, configured to adjust the axial position of the balloon catheter 20 relative to the outer shaft 18, for example for fine positioning at the implantation site. The handle can include additional knobs to control additional components of the delivery apparatus 12. Further details on the delivery apparatus 12 can be found in PCT Application No. PCT/US2021/047056, which is incorporated by reference herein.

A prosthetic valve 30 can be carried by the delivery apparatus 12 during delivery in a crimped state, and expanded, for example by balloon inflation, to secure it in a native heart valve annulus (such as an aortic annulus) or against a previously implanted prosthetic valve (for example, during valve-in-valve implantation procedures). In some examples, the balloon 22 is secured to a distal end portion of the balloon catheter 20 at its proximal end, while the balloon's distal end can be coupled, directly or indirectly, to another component of the delivery apparatus 12, such as the nosecone 24 or nosecone shaft.

Balloon 22 is configured to transition between a deflated and inflated states. Upon reaching the site of implantation, the balloon 22 can be inflated to radially expand the prosthetic valve 30. Once the prosthetic valve 30 is expanded to its functional diameter within a native annulus, the balloon 22 can be deflated, and the delivery apparatus 12 can be retrieved from the patient's body.

In some examples, the delivery apparatus 12 with the prosthetic valve 30 assembled thereon, can be packaged in a sterile package that can be supplied to end users for storage and eventual use. In some examples, when the leaflets of the prosthetic valve are made from, or include at least an inner core made from, bovine pericardium tissue or other natural or synthetic tissues, the leaflets can be treated during the manufacturing process so that they are completely or substantially dehydrated and can be stored in a partially or fully crimped state without a hydrating fluid. In this manner, the package containing the prosthetic valve and the delivery apparatus, can be free of any liquid. Methods for treating tissue leaflets for dry storage are disclosed in U.S. Pat. Nos. 8,007,992 and 8,357,387, both of which documents are incorporated herein by reference.

A prosthetic valve 100 may be expanded against a calcified aortic annulus, requiring it to overcome the relatively increased rigidity of the calcified tissue during expansion. One of the factors known to affect the radial force exerted by the frame 102 of a valve 100 on the surrounding anatomy is the number of cells 150 in corresponding cell rows, wherein a greater number of cells 150 (i.e., a higher cell density) will result in a greater radial force during expansion. Thus, conventional valve frames 102 may be provided with a relatively large number of cells 150 to increase the radial force to overcome the resistance of the calcified annular pathologies.

Some prosthetic valves may have an overall axial length, in their expanded state, that can place the upper or first cell row 152 at the level of the coronary ostia. For example, such valves 100 can be designed to have their outflow apices 168 contacting or being placed in the vicinity of the sinuses or the Sinotubular Junction (STJ) when expanded at the site of implantation. In some instances, a patient may require implantation of a coronary stent of other procedure that requires access to a coronary artery after prosthetic valve implantation. For such instances, a physician may need to access the coronary artery through the opening defined by a cell 150 of the first cell row 152 facing the coronary ostium. While the cells 150 of the first cell row 152 of the prosthetic valve 100 illustrated, for example, in FIGS. 1A-1C, may have a height (measured in the axial direction) which is greater than the height of other cell rows 154. 156, due to the axial frame members 130 interconnecting the first 112 and second 114 rungs of angled struts, their width (measured in the circumferential direction, such as between two immediately adjacent axial frame members 130) is identical to the width of all other cells 150. Since the number of cells can be chosen to be relatively large to provide sufficient radial force during expansion against the native annulus, this may result in a cell width that can compromise the ability for future access into the coronary arteries or perfusion through the frame 102 to the coronary arteries during the diastolic phase of the cardiac cycle.

Taking advantage of the fact that increased radial force during valve expansion is required primarily at the region of the native annulus, corresponding to the lower portion (i.e., distal or inflow portion) of the frame, an upper cell row of frame configurations disclosed herein can include wider cells than those of the other cell rows. The width of the wider cells in the first cell row can be configured to be larger than the outer diameter of a selected coronary catheter (e.g., a 6 Fr coronary catheter).

FIGS. 3A-15 show various frame configurations of exemplary prosthetic valves disclosed herein, comprising cells in the first cell row that are wider, in the circumferential direction, than cells of some or all of the subsequent cell rows, optionally with more than two angled struts extending between immediately adjacent axial frame members along one or both rungs interconnected by the axial frame members. The frames (or portions thereof) are shown throughout FIGS. 3A-15 in a flattened state and without soft components (such as leaflets and skirts) for clarity. Moreover, it is to be understood that the number of cell rows in the illustrated exemplary frames can vary, and that the same frames can include additional rows of cells relative to the illustrated examples.

FIG. 3A shows a frame 202 of an exemplary prosthetic valve 200, which is similar in structure and function to frame 102 of prosthetic valve 100 disclosed above with respect to FIGS. 1A-1C, with like numbers referring to like components, except that each of the first rung 212 and second rung 214 of the frame 202 comprises three angled struts 210 extending between immediately adjacent axial frame members 230. The frame 202 can include a single axial strut 232 disposed between immediately adjacent commissure support 234. The commissure supports 234 are attached at their upper ends 246 to upper junctions 264 of the first rung 212 of angled struts, which are also the outflow apices 268 of the illustrated exemplary frame 202, and are attached at their lower ends 248 to lower junctions 266 of the second rung 214 of angled struts.

Each cell 250 of the first cell row 252 can span a width of 1.5 cells 250 of the second cell row 254 and/or of the third cell row 256. Thus, compared to cells of the first cell row 152 of prosthetic valve 100, cells 250 of the first cell row 252 can have an increased width (in the circumferential direction), thereby providing a larger opening for blood flow and/or coronary access. In some examples, the first cell row 252 includes six cells 250, while any subsequent cell row, such as the second cell row 254 and/or third cell row 256 can include nine cells 250.

The axial struts 232 are attached at their upper ends 246 to lower junctions 266 of the first rung 212 of angled struts, and are attached at their lower ends 248 to upper junctions 264 of the second rung 214 of angled struts. This results in commissure supports 334 having a length L_{C} greater than the length L_{N} of the axial struts 332. In some examples, a frame 202 includes three commissure supports 234 and three axial struts 232 alternately arrange around the circumference of the first cell row 252.

In some examples, a commissure support can include an upper strut portion extending from the commissure window to the upper end of the commissure support, and/or a lower strut portion extending from the commissure window to the lower end of the commissure support. Since the total length L_{C} of a commissure support, such as commissure support 234 of frame 202, can be greater than the length L_{N} of an axial strut 232, an upper strut portion 242 and/or a lower strut portion 244 thereof can provide added length to the commissure support 234 and allow the upper strut portion 242 and/or a lower strut portion 244 to bridge the larger distance between the upper junction 264 of the first rung 212 and the lower junction 266 of the second rung 214, while the dimensions of the commissure window 236 can be relatively similar to the dimensions of the commissure window 136 of prosthetic valve 100 described above and illustrated in FIGS. 1A-1C.

Various exemplary implementations for prosthetic valves and components thereof can be referred to, throughout the specification, with superscripts, for ease of explanation of features that refer to such exemplary implementations. It is to be understood, however, that any reference to structural or functional features of any valve, frame or component, without a superscript, refer to these features being commonly shared by all specific exemplary implementations that can be also indicated by superscripts. In contrast, features emphasized with respect to an exemplary implementation of device or component, including prosthetic valves and frames thereof, referred to with a superscript, may be optionally shared by some but not necessarily all other exemplary implementations.

For example, frame 202^{a} illustrated in FIG. 3A is an exemplary implementation of frame 202, and thus can include any of the features described for frame 202 throughout the current disclosure, except that the commissure support 234^{a} of frame 202^{a}comprises both an upper strut portion 242 extending between its upper end 246 and the commissure window 236, and a lower strut portion 244 extending between its lower end 248 and the commissure window 236. In some examples, the lengths of the upper 242 and lower 244 strut portions are substantially similar, as illustrated in FIG. 3A. In some examples, the lengths of the upper 242 and lower 244 strut portion can be different from each other. For example, the upper strut portion 242 can be shorter than the lower strut portion 244, which will place the commissure window 236 closer to the upper end 246 of the respective commissure support 234^{a}, or the upper strut portion 242 can be longer than the lower strut portion 244, which will place the commissure window 236 closer to the lower end 248 of the respective commissure support 234^{a}.

FIG. 3B shows an exemplary frame 202^{b}, which is similar to any example described herein for frame 202, except that the commissure support 234^{b} of frame 202^{b} comprises an upper strut portion 242 extending between its upper end 246 and the commissure window 236, while being devoid of a lower strut portion, such that the lower end 248 of the commissure support 234^{b} can be defined by the lower end of the commissure window 236.

It is to be understood that additional configurations of the commissure support 234 are contemplated, such as a commissure support 234 comprising a lower strut portion 244 while being devoid of an upper strut portion.

As further shown in FIGS. 3A-3B, at least some of the upper junctions 264 of the second rung 214 of angled struts are free apices 272 that are distal to the outflow apices 268 and are unattached to axial frame members 230 or to any other struts 208 besides the two angled struts 210 of the second rung 214 diverging from each respective free apex 272. In some examples, each cell 250 of the first cell row 252 comprises a free apex 272 of the second rung 214 of angled struts.

FIG. 4A show a frame 302 of an exemplary prosthetic valve 300, which is similar in structure and function to frame 202 of prosthetic valve 200 disclosed above with respect to FIGS. 3A-3B, with like numbers referring to like components, except that the commissure supports 334 are attached at their upper ends 346 to lower junctions 366 of the first rung 312 of angled struts, and are attached at their lower ends 348 to upper junctions 364 of the second rung 314 of angled struts. The frame 302 further differs from frame 202 in that the axial struts 332 are attached at their upper ends 346 to upper junctions 364 of the first rung 312 of angled struts, which are also the outflow apices 368 of the illustrated exemplary frame 302, and are attached at their lower ends 348 to lower junctions 366 of the second rung 314 of angled struts. This results in axial struts 332 having a length L_{N} greater than the length L_{C} of the commissure supports 334.

Similar to frame 202, each of the first rung 312 and second rung 314 of the frame 302 comprises three angled struts 310 extending between immediately adjacent axial frame members 330. Each cell 350 of the first cell row 352 can span a width of 1.5 cells 350 of the second cell row 354 and/or of the third cell row 356. In some examples, the first cell row 352 includes six cells 350, while any subsequent cell row, such as the second cell row 354 and/or third cell row 356 can include nine cells 350. The frame 302 can include a single axial strut 332 disposed between immediately adjacent commissure support 334. In some examples, a frame 302 includes three commissure supports 334 and three axial struts 332 alternately arrange around the circumference of the first cell row 352. The second rung 314 can include free apices 372 distal to the outflow apices 368. In some examples, each cell 350 of the first cell row 352 can include a free apex 372 of the second rung 314 of angled struts.

In some examples, commissure supports 334 of frame 302 can include upper strut portions 342 and/or lower strut portions 344. Inclusion of upper 342 or lower 344 strut portions, even when the commissure supports 334 are relatively shorter than the axial struts 332, can advantageously provide added length to commissure supports 334 (while proportionally elongating even further the axial struts 332), to allow for increased height of the cells 350 of the first cell row 352, without the need to modify the valvular structure or commissures thereof.

FIG. 4A shows an exemplary frame 302^{a}, which is similar to any example described herein for frame 302, except that the commissure support 334^{a} of frame 302^{a} comprises an upper strut portion 342 extending between its upper end 346 and the commissure window 336, while being devoid of a lower strut portion, such that the lower end 348 of the commissure support 334^{a} can be defined by the lower end of the commissure window 336.

FIG. 4B shows an exemplary frame 302^{b}, which is similar to any example described herein for frame 302, except that the commissure support 334^{b} of frame 302^{b} comprises both an upper strut portion 342 extending between its upper end 346 and the commissure window 336, and a lower strut portion 344 extending between its lower end 348 and the commissure window 336. In some examples, the lengths of the upper 342 and lower 344 strut portions are substantially similar, as illustrated in FIG. 4B. In some examples, the lengths of the upper 342 and lower 344 strut portion can be different from each other. For example, the upper strut portion 342 can be shorter than the lower strut portion 344, which will place the commissure window 336 closer to the upper end 346 of the respective commissure support 334^{b}, or the upper strut portion 342 can be longer than the lower strut portion 344, which will place the commissure window 336 closer to the lower end 348 of the respective commissure support 334^{b}.

FIG. 4C shows an exemplary frame 302^{c}, which is similar to any example described herein for frame 302, except that the commissure support 334^{c} of frame 302^{c} comprises a lower strut portion 344 extending between its lower end 348 and the commissure window 336, while being devoid of an upper strut portion, such that the upper end 346 of the commissure support 334^{c} can be defined by the upper end of the commissure window 336.

FIG. 5A show a frame 402 of an exemplary prosthetic valve 400, which is similar in structure and function to any of frame 202 of prosthetic valve 200 or frame 302 of prosthetic valve 300 disclosed above with respect to FIGS. 3A-4C, with like numbers referring to like components, except that the commissure supports 434 are attached at their upper ends 446 to lower junctions 466 of the first rung 412 of angled struts, and are attached at their lower ends 448 to lower junctions 466 of the second rung 414 of angled struts. Furthermore, the axial struts 432 are attached at their upper ends 446 to upper junctions 464 of the first rung 412 of angled struts, which are also the outflow apices 468 of the illustrated exemplary frame 402, and are attached at their lower ends 448 to upper junctions 464 of the second rung 414 of angled struts. Thus, the commissure supports 434 and axial struts 432 can have substantially similar lengths (i.e., L_{C}=L_{N}).

Similar to frames 202 and 302, each of the first rung 412 and second rung 414 of the frame 402 comprises three angled struts 410 extending between immediately adjacent axial frame members 430. Each cell 450 of the first cell row 452 can span a width of 1.5 cells 450 of the second cell row 454 and/or of the third cell row 456. In some examples, the first cell row 452 includes six cells 450, while any subsequent cell row, such as the second cell row 454 and/or third cell row 456 can include nine cells 450. The frame 402 can include a single axial strut 432 disposed between immediately adjacent commissure support 434. In some examples, a frame 402 includes three commissure supports 434 and three axial struts 432 alternately arrange around the circumference of the first cell row 452. The second rung 414 can include free apices 472 distal to the outflow apices 468. In some examples, each cell 450 of the first cell row 452 can include a free apex 472 of the second rung 414 of angled struts.

In some examples, commissure supports 434 of frame 402 can include an upper strut portions 442 and/or lower strut portions 444. FIG. 5A shows an exemplary frame 402^{a}, which is similar to any example described herein for frame 402, except that the commissure support 434^{a} of frame 402^{a} comprises an upper strut portion 442 extending between its upper end 446 and the commissure window 436, while being devoid of a lower strut portion, such that the lower end 448 of the commissure support 434^{a} can be defined by the lower end of the commissure window 436.

FIG. 5B shows an exemplary frame 402^{b}, which is similar to any example described herein for frame 402, except that the commissure support 434^{b} of frame 402^{b} comprises both an upper strut portion 442 extending between its upper end 446 and the commissure window 436, and a lower strut portion 444 extending between its lower end 448 and the commissure window 436. In some examples, the lengths of the upper 442 and lower 444 strut portions are substantially similar, as illustrated in FIG. 5B. In some examples, the lengths of the upper 442 and lower 444 strut portion can be different from each other. For example, the upper strut portion 442 can be shorter than the lower strut portion 444, which will place the commissure window 436 closer to the upper end 446 of the respective commissure support 434^{b}, or the upper strut portion 442 can be longer than the lower strut portion 444, which will place the commissure window 436 closer to the lower end 448 of the respective commissure support 434^{b}.

FIG. 5C shows an exemplary frame 402^{c}, which is similar to any example described herein for frame 402, except that the commissure support 434^{c} of frame 402^{c} comprises a lower strut portion 444 extending between its lower end 448 and the commissure window 436, while being devoid of an upper strut portion, such that the upper end 446 of the commissure support 434^{c} can be defined by the upper end of the commissure window 436.

FIG. 6A show a frame 502 of an exemplary prosthetic valve 500, which is similar in structure and function to frame 402 of prosthetic valve 400 disclosed above with respect to FIGS. 5A-5C, with like numbers referring to like components, except that the commissure supports 534 are attached at their upper ends 546 to upper junctions 564 of the first rung 512 of angled struts, which are also the outflow apices 568 of the illustrated exemplary frame 502, and are attached at their lower ends 548 to upper junctions 564 of the second rung 514 of angled struts. Furthermore, the axial struts 532 are attached at their upper ends 546 to lower junctions 566 of the first rung 512 of angled struts, and are attached at their lower ends 548 to lower junctions 566 of the second rung 514 of angled struts.

Thus, similarly to frame 402, the commissure supports 534 and axial struts 532 can have substantially similar lengths (i.e., L_{C}=L_{N}). Furthermore, each of the first rung 512 and second rung 514 of the frame 502 comprises three angled struts 510 extending between immediately adjacent axial frame members 530. Each cell 550 of the first cell row 552 can span a width of 1.5 cells 550 of the second cell row 554 and/or of the third cell row 556. In some examples, the first cell row 552 includes six cells 550, while any subsequent cell row, such as the second cell row 554 and/or third cell row 556 can include nine cells 550. The frame 502 can include a single axial strut 532 disposed between immediately adjacent commissure support 534. In some examples, a frame 502 includes three commissure supports 534 and three axial struts 532 alternately arrange around the circumference of the first cell row 552. The second rung 514 can include free apices 572 distal to the outflow apices 568. In some examples, each cell 550 of the first cell row 552 can include a free apex 572 of the second rung 514 of angled struts.

In some examples, commissure supports 534 of frame 502 can include an upper strut portions 542 and/or lower strut portions 544. FIG. 6A shows an exemplary frame 502^{a}, which is similar to any example described herein for frame 502, except that the commissure support 534^{a} of frame 502^{a} comprises an upper strut portion 542 extending between its upper end 546 and the commissure window 536, while being devoid of a lower strut portion, such that the lower end 548 of the commissure support 534^{a} can be defined by the lower end of the commissure window 536.

FIG. 6B shows an exemplary frame 502^{b}, which is similar to any example described herein for frame 502, except that the commissure support 534^{b} of frame 502^{b} comprises both an upper strut portion 542 extending between its upper end 546 and the commissure window 536, and a lower strut portion 544 extending between its lower end 548 and the commissure window 536. In some examples, the lengths of the upper 542 and lower 544 strut portions are substantially similar, as illustrated in FIG. 6B. In some examples, the lengths of the upper 542 and lower 544 strut portion can be different from each other. For example, the upper strut portion 542 can be shorter than the lower strut portion 544, which will place the commissure window 536 closer to the upper end 546 of the respective commissure support 534^{b}, or the upper strut portion 542 can be longer than the lower strut portion 544, which will place the commissure window 536 closer to the lower end 548 of the respective commissure support 534^{b}.

FIG. 6C shows an exemplary frame 502^{c}, which is similar to any example described herein for frame 502, except that the commissure support 534^{c} of frame 502^{c} comprises a lower strut portion 544 extending between its lower end 548 and the commissure window 536, while being devoid of an upper strut portion, such that the upper end 546 of the commissure support 534^{c} can be defined by the upper end of the commissure window 536.

FIG. 7 shows a portion of a frame 602 of an exemplary prosthetic valve 600, which is similar in structure and function to frame 202 of prosthetic valve 200 disclosed above with respect to FIGS. 3A-3B, with like numbers referring to like components, except that apices of the frame 602, such as any of the outflow apices 668, inflow apices 670, and free apices 672, comprise arcuate regions 676 defined between upper curved surfaces 678 axially facing the outflow end 704 and lower curved surfaces 680 axially facing the inflow end 606. In some examples, the arcuate regions 676 of any of the apices 668, 670, 672 are thinner than the remainder of the corresponding angled struts 610 diverging therefrom.

The term "width", as used herein with respect to a strut or an apex, refers to a dimension of a strut or an arcuate region of an apex measured between opposing locations on opposing surfaces of the strut or apex that extend between the radially facing inner and outer surfaces of the strut or apex (relative to the central longitudinal axis Ca), respectively. For example, an arcuate region 676 has a width W_{A} defined between its upper 678 and lower 680 curved surfaces, which is less than a width W_{S} of a strut continuously extending therefrom. The arcuate region 676 can have a uniform width W_{A} along a length L_{A}, which can be in the range of 10%-25% of a length L_{S} of a corresponding angled strut 610 diverging therefrom, as shown in FIG. 7, wherein the length L_{S} is inclusive of half the arcuate region 676.

In the example illustrated in FIG. 7, each outflow apex 668 of the first rung 612 comprises an upwardly convex-shaped upper curved surface 678 and an opposing lower curved surface 680 that can form an inner depression of the apex 668, each free apex 672 of the second rung 614 comprises an upwardly convex-shaped upper curved surface 678 and an opposing lower curved surface 680 that can form an inner depression of the apex 672, and each inflow apex 670 of the fourth rung 618 (or another rung forming a lowermost rung of angled struts for frames that include other numbers of strut rungs) comprises a downwardly convex-shaped lower curved surface 680 and an opposing upper curved surface 678 that can form an inner depression of the apex 670.

In some examples, the arcuate region 676 of an outflow apex 668 or free apex 672 can have a radius of curvature along the upper curved surface 678, and the arcuate region 676 of an inflow apex 670 can have a radius of curvature along the lower curved surface 680. In some examples, the radius of curvature of arcuate region 676 can be greater than 1 mm. In some examples, the radius of curvature of arcuate region 676 can be in a range of 1-20 mm, 3-16 mm, or 8-14 mm. In some examples, the radius of curvature of arcuate region 676 can be greater than 10 mm. Additional details and examples of frames for prosthetic valves that include apices with arcuate regions can be found in PCT Application No. PCT/US2022/025687, which is incorporated by reference herein.

Similar to frame 202, each of the first rung 612 and second rung 614 of the frame 602 comprises three angled struts 610 extending between immediately adjacent axial frame members 630. The commissure supports 634 are longer than the axial struts 632 (i.e., L_{C}>L_{N}). Each cell 650 of the first cell row 652 can span a width of 1.5 cells 650 of the second cell row 654 and/or of the third cell row 656. In some examples, the first cell row 652 includes six cells 650, while any subsequent cell row, such as the second cell row 654 and/or third cell row 656 can include nine cells 650. The frame 602 can include a single axial strut 632 disposed between immediately adjacent commissure support 634. In some examples, a frame 602 includes three commissure supports 634 and three axial struts 632 alternately arranged around the circumference of the first cell row 652.

In some examples, commissure supports 634 of frame 602 can include upper strut portions 642 and/or lower strut portions 644. In some examples, a commissure support 634 of frame 602 includes a lower strut portion 644 extending between its lower end 648 and the commissure window 636, while being devoid of an upper strut portion, as shown in the example illustrated in FIG. 7. In some examples, a commissure support 634 of frame 602 includes an upper strut portion 642 extending between its upper end 646 and the commissure window 636, while being devoid of a lower strut portion, in a similar manner to that shown for commissure supports 234^{b}in FIG. 3B. In some examples, a commissure support 634 of frame 602 includes both an upper strut portion 642 extending between its upper end 646 and the commissure window 636, and a lower strut portion 644 extending between its lower end 648 and the commissure window 636, in a similar manner to that shown for commissure supports 234^{a} in FIG. 3A.

FIG. 8 shows a portion of a frame 702 of an exemplary prosthetic valve 700, which is similar in structure and function to frame 502 of prosthetic valve 500 disclosed above with respect to FIGS. 6A-6C, with like numbers referring to like components, except that apices of the frame 702, such as any of the outflow apices 768, inflow apices 770, and free apices 772, comprise arcuate regions 776 defined between upper curved surfaces 778 axially facing the outflow end 704 and lower curved surfaces 780 axially facing the inflow end 706. The arcuate regions 776 can be similar to arcuate regions 676 described above with respect to frame 602, and may have a thickness T_{A} along a length L_{A} which is less than the thickness T_{S} of corresponding angled struts 710 diverging therefrom.

In the example illustrated in FIG. 8, each outflow apex 768 of the first rung 712 comprises an upwardly convex-shaped upper curved surface 778 and an opposing lower curved surface 780 that can form an inner depression of the apex 768, each free apex 772 of the second rung 714 comprises an upwardly convex-shaped upper curved surface 778 and an opposing lower curved surface 780 that can form an inner depression of the apex 772, and each inflow apex 770 of the fourth rung 718 (or another rung forming a lowermost rung of angled struts for frames that include other numbers of strut rungs) comprises a downwardly convex-shaped lower curved surface 780 and an opposing upper curved surface 778 that can form an inner depression of the apex 770.

Similar to frame 502, each of the first rung 712 and second rung 714 of the frame 702 comprises three angled struts 710 extending between immediately adjacent axial frame members 730. The commissure supports 734 and axial struts 732 can have substantially similar lengths (i.e., L_{C}=L_{N}). Each cell 750 of the first cell row 752 can span a width of 1.5 cells 750 of the second cell row 754 and/or of the third cell row 756. In some examples, the first cell row 752 includes six cells 750, while any subsequent cell row, such as the second cell row 754 and/or third cell row 756 can include nine cells 750. The frame 702 can include a single axial strut 732 disposed between immediately adjacent commissure support 734. In some examples, a frame 702 includes three commissure supports 734 and three axial struts 732 alternately arrange around the circumference of the first cell row 752.

In some examples, commissure supports 734 of frame 702 can include upper strut portions 742 and/or lower strut portions 744. In some examples, a commissure support 734 of frame 702 includes a lower strut portion 744 extending between its lower end 748 and the commissure window 736, while being devoid of an upper strut portion, as shown in the example illustrated in FIG. 8. In some examples, a commissure support 734 of frame 702 includes an upper strut portion 742 extending between its upper end 746 and the commissure window 736, while being devoid of a lower strut portion, in a similar manner to that shown for commissure supports 534^{a} in FIG. 6A. In some examples, a commissure support 734 of frame 702 includes both an upper strut portion 742 extending between its upper end 746 and the commissure window 736, and a lower strut portion 744 extending between its lower end 748 and the commissure window 736, in a similar manner to that shown for commissure supports 734^{b} in FIG. 6B.

While arcuate regions 676 and 776 are described above with respect to frames 602 and 702 illustrated in FIGS. 7 and 8, respectively, it is to be understood that apices of any of the frames 202, 302, 402 or 502, including any of their respective outflow apices 268, 368, 468 or 568, inflow apices 270, 370, 470 or 570, and/or free apices 272, 372, 472 or 572, can include similar arcuate regions defined between corresponding upper and lower curved surfaces.

FIG. 9 shows a portion of a frame 802 of an exemplary prosthetic valve 800, which is similar in structure and function to frame 402 of prosthetic valve 400 disclosed above with respect to FIGS. 5A-5C, with like numbers referring to like components, except that the commissure supports 834 are devoid of upper or lower strut portions. As illustrated, the axial struts 832 are attached at their upper ends 846 to upper junctions 864 of the first rung 812 of angled struts, which are also the outflow apices 868 of the illustrated exemplary frame 802, and are attached at their lower ends 848 to upper junctions 864 of the second rung 814 of angled struts. Furthermore, the commissure supports 834 are attached at their upper ends 846 to lower junctions 866 of the first rung 812 of angled struts, and are attached at their lower ends 848 to lower junctions 866 of the second rung 814 of angled struts.

Thus, similarly to frame 402, the commissure supports 834 and axial struts 832 can have substantially similar lengths (i.e., L_{C}=L_{N}). However, since the commissure supports 834 are devoid of upper or lower strut portions, the commissure windows 836 extend along the entire length L_{C}, such that the upper end 846 of the commissure support 834 can be defined by the upper end of the commissure window 836, and the lower end 848 of the commissure support 834 can be defined by the lower end of the commissure window 836.

Each of the first rung 812 and second rung 814 of the frame 802 comprises three angled struts 810 extending between immediately adjacent axial frame members 830. Each cell 850 of the first cell row 852 can span a width of 1.5 cells 850 of the second cell row 854 and/or of the third cell row 856. In some examples, the first cell row 852 includes six cells 850, while any subsequent cell row, such as the second cell row 854 and/or third cell row 856 can include nine cells 850. The frame 802 can include a single axial strut 832 disposed between immediately adjacent commissure support 834. In some examples, a frame 802 includes three commissure supports 834 and three axial struts 832 alternately arrange around the circumference of the first cell row 852. The second rung 814 can include free apices 872 distal to the outflow apices 868. In some examples, each cell 850 of the first cell row 852 can include a free apex 872 of the second rung 814 of angled struts.

In some examples, apices of the frame 802, such as any of the outflow apices 868, inflow apices 870, and free apices 872, can include arcuate regions 876 defined between upper curved surfaces 878 lower curved surfaces 880, which can be similar to arcuate regions 676 described above with respect to frame 602, and may have a thickness T_{A} along a length L_{A} which is less than the thickness T_{S} of corresponding angled struts 810 diverging therefrom.

FIG. 10 shows a portion of a frame 902 of an exemplary prosthetic valve 900, which is similar in structure and function to frame 502 of prosthetic valve 500 disclosed above with respect to FIGS. 6A-6C, with like numbers referring to like components, except that the commissure supports 934 are devoid of upper or lower strut portions. As illustrated, the commissure supports 934 are attached at their upper ends 946 to lower junctions 966 of the first rung 912 of angled struts, and are attached at their lower ends 948 to lower junctions 966 of the second rung 914 of angled struts. Furthermore, the axial struts 932 are attached at their upper ends 946 to upper junctions 964 of the first rung 912 of angled struts, which are also the outflow apices 968 of the illustrated exemplary frame 902, and are attached at their lower ends 948 to upper junctions 964 of the second rung 914 of angled struts.

Thus, similarly to frame 502, the commissure supports 934 and axial struts 932 can have substantially similar lengths (i.e., L_{C}=L_{N}). However, since the commissure supports 934 are devoid of upper or lower strut portions, the commissure windows 936 extend along the entire length L_{C}, such that the upper end 946 of the commissure support 934 can be defined by the upper end of the commissure window 936, and the lower end 948 of the commissure support 934 can be defined by the lower end of the commissure window 936.

Each of the first rung 912 and second rung 914 of the frame 902 comprises three angled struts 910 extending between immediately adjacent axial frame members 930. Each cell 950 of the first cell row 952 can span a width of 1.5 cells 950 of the second cell row 954 and/or of the third cell row 956. In some examples, the first cell row 952 includes six cells 950, while any subsequent cell row, such as the second cell row 954 and/or third cell row 956 can include nine cells 950. The frame 902 can include a single axial strut 932 disposed between immediately adjacent commissure support 934. In some examples, a frame 902 includes three commissure supports 934 and three axial struts 932 alternately arrange around the circumference of the first cell row 952. The second rung 914 can include free apices 972 distal to the outflow apices 968. In some examples, each cell 950 of the first cell row 952 can include a free apex 972 of the second rung 914 of angled struts.

In some examples, apices of the frame 902, such as any of the outflow apices 968, inflow apices 970, and free apices 972, can include arcuate regions 976 defined between upper curved surfaces 978 lower curved surfaces 980, which can be similar to arcuate regions 676 described above with respect to frame 602, and may have a thickness T_{A} along a length L_{A} which is less than the thickness T_{S} of corresponding angled struts 910 diverging therefrom.

FIG. 11A shows a portion of a frame 1002 of an exemplary prosthetic valve 1000, which is similar in structure and function to frame 102 of prosthetic valve 100 disclosed above with respect to FIGS. 1A-1C, with like numbers referring to like components, except that the first rung 1012 comprises two angled struts 1010 extending between immediately adjacent axial frame members 1030, while the second rung 1014 comprises three angled struts 1010 extending between immediately adjacent axial frame members 1030. The commissure supports 1034 are attached at their upper ends 1046 to lower junctions 1066 of the first rung 1012 of angled struts, and are attached at their lower ends 1048 to lower junctions 1066 of the second rung 1014 of angled struts. The axial struts 1032 are attached at their upper ends 1046 to lower junctions 1066 of the first rung 1012 of angled struts, and are attached at their lower ends 1048 to upper junctions 1064 of the second rung 1014 of angled struts. Thus, the commissure supports 1034 can be longer than the axial struts 1032 (i.e., L_{C}>L_{N}).

Each cell 1050 of the first cell row 1052 can span a width of 1.5 cells 1050 of the second cell row 1054 and/or of the third cell row 1056. In some examples, the first cell row 1052 includes six cells 1050, while any subsequent cell row, such as the second cell row 1054 and/or third cell row 1056 can include nine cells 1050. The frame 1002 can include a single axial strut 1032 disposed between immediately adjacent commissure support 1034.

In some examples, commissure supports 1034 of frame 1002 can include upper strut portions 1042 and/or lower strut portions 1044. In some examples, a commissure support 1034 of frame 1002 includes a lower strut portion 1044 extending between its lower end 1048 and the commissure window 1036, while being devoid of an upper strut portion, as shown in the example illustrated in FIG. 11A. In some examples, a commissure support 1034 of frame 1002 includes an upper strut portion 1042 extending between its upper end 1046 and the commissure window 1036, while being devoid of a lower strut portion, in a similar manner to that shown for commissure supports 234^{b} in FIG. 3B. In some examples, a commissure support 1034 of frame 1002 includes both an upper strut portion 1042 extending between its upper end 1046 and the commissure window 1036, and a lower strut portion 1044 extending between its lower end 1048 and the commissure window 1036, in a similar manner to that shown for commissure supports 234^{a} in FIG. 3A. In some examples, a commissure support 1034 of frame 1002 can be devoid of upper or lower strut portions, in a similar manner to that shown for commissure supports 834 in FIG. 9.

The length L_{S} of an angled strut 1010 of the first rung 1012 is greater than the length L_{S} of an angled strut 1010 of the second rung 1014, and/or any other rung of the frame 1002, such as the third rung 1016 and the fourth rung 1018. In some examples, the width W_{S} of an angled struts 1010 of the first rung 1012 can be larger than the width Ws of angled struts 1010 of the second rung 1014, and/or angled struts 1010 of any other rung of the frame 1002, such as the third rung 1016 and the fourth rung 1018.

In some examples, apices of the frame 1002, such as any of the outflow apices 1068, inflow apices 1070, and/or free apices 1072, can include arcuate regions 1076 defined between upper curved surfaces 1078 lower curved surfaces 1080, which can be similar to arcuate regions 676 described above with respect to frame 602, and may have a thickness T_{A} along a length L_{A} which is less than the thickness T_{S} of corresponding angled struts 1010 diverging therefrom.

In some examples, some of the apices of any frame disclosed herein can include arcuate regions defined between upper and lower curved surfaces, while other apices can be formed without such arcuate regions. FIG. 11A shows an exemplary frame 1002^{a}, which is similar to any example described herein for frame 1002, except that the outflow apices 1068 along the first rung 1012 comprise arcuate regions 1076, while free apices 1072 along the second rung 1014 can be in the form of "pointed" apices, devoid of arcuate regions.

While the inflow apices 1070 are in FIG. 11A to be also devoid of arcuate regions, it is to be understood that in some examples, both the inflow and outflow apices of the frame can have arcuate regions, while other apices, such as free apices 1072, can be formed without arcuate regions. While frames disclosed herein are illustrated to have all apices either with arcuate regions (as illustrated, for example, in FIGS. 7-10 and 12B-15) or without arcuate regions (as illustrated, for example, in FIGS. 2A-6C and 13), it is to be understood that any frame disclosed herein can include, in some examples, any combination of some apices that include arcuate regions, and some apices which are devoid of arcuate regions.

FIG. 11B shows an exemplary frame 1002^{b}, which is similar to any example described herein for frame 1002, except that all apices, including the outflow apices 1068 along the first rung 1012, the free apices 1072 along the second rung 1014, and the inflow apices 1070 along the fourth rung 1018, include arcuate regions 1076. In some cases, the free apices 1072 along the second rung 1014 can interact with the leaflets of the prosthetic heart valve. For example, when the leaflets are secured to the frame, the free apices 1072 can be disposed at a level (or axial height) of a portion of the leaflets that open and close during operation of the prosthetic valve.

Thus, in some instances, when the leaflets are in an open state when implanted in a patient, the leaflets can contact the free apices 1072. In such cases, it is beneficial for the free apices to have a arcuate regions with curved outer surfaces, as illustrated in FIGS. 7-10 and 11B. The curved outer surface is more atraumatic and may not interfere with the leaflets of the prosthetic valve as the leaflets open and close during operation of the prosthetic valve. Thus, a long term durability of the leaflets can be increased.

FIG. 11C shows an exemplary frame 1002^{c}, which is similar to any example described herein for frame 1002, except that each outflow apex 1068 is circumferentially aligned with a lower junction 1066 of the second rung 1014, which is not attached to any axial frame member 1030. This results in each pair of angled struts 1010 of the first rung 1012, extending between immediately adjacent axial frame members 1030, including one angled strut extending between the outflow apex 1068 and the upper end 1046 of the corresponding commissure support 1034, along a strut length L_{S1}, and one angled strut extending between the outflow apex 1068 and the upper end 1046 of the corresponding axial strut 1032, along a length L_{S2}, such that the length L_{S1} is greater than the length L_{S2}. This configuration advantageously allows the frame 1002^{c}to be properly crimped, without deforming any of the struts due to misalignment between junctions of the first and second rungs.

FIG. 12A shows a portion of a frame 1102 of an exemplary prosthetic valve 1100, which is similar in structure and function to frame 1002 of prosthetic valve 1000 disclosed above with respect to FIGS. 11A-11B, with like numbers referring to like components, except that while the first rung 1112 comprises two angled struts 1110 extending between immediately adjacent axial frame members 1130, the second rung 1114 comprises four angled struts 1110 extending between immediately adjacent axial frame members 1130. As further illustrated, each lower junction 1166 of the first rung 1112 is circumferentially aligned with one of the upper junctions 1164 of the second rung 1114. In contrast to frame 1002, all axial frame members 1130 of the frame 1102, including the axial struts 1132 and commissure supports 1134, are attached at their upper ends 1146 to upper junctions 1164 of the first rung 1112 of angled struts, which are also the outflow apices 1168 of the illustrated exemplary frame 1102, and are attached at their lower ends 1148 to upper junctions 1164 of the second rung 1114 of angled struts. Thus, the commissure supports 1134 and the axial struts 1132 can have substantially similar lengths (i.e., L_{C}=L_{N}).

Each cell 1150 of the first cell row 1152 can span a width of two cells 1150 of the second cell row 1154 and/or of the third cell row 1156. In some examples, the first cell row 1152 includes six cells 1150, while any subsequent cell row, such as the second cell row 1154 and/or third cell row 1156 can include twelve cells 1150. The frame 1102 can include a single axial strut 1132 disposed between immediately adjacent commissure support 1134.

In some examples, commissure supports 1134 of frame 1102 can include upper strut portions 1142 and/or lower strut portions 1144. In some examples, a commissure support 1134 of frame 1102 includes an upper strut portion 1142 extending between its upper end 1146 and the commissure window 1136, while being devoid of a lower strut portion, as shown in the example illustrated for frame 1102^{a} in FIG. 12A. In some examples, a commissure support 1134 of frame 1102 includes a lower strut portion 1144 extending between its lower end 1148 and the commissure window 1136, while being devoid of an upper strut portion, in a similar manner to that shown for commissure supports 334^{c} in FIG. 4C. In some examples, a commissure support 1134 of frame 1102 includes both an upper strut portion 1142 extending between its upper end 1146 and the commissure window 1136, and a lower strut portion 1144 extending between its lower end 1 and the commissure window 1036, in a similar manner to that shown for commissure supports 234^{a} in FIG. 3A. In some examples, a commissure support 1134 of frame 1102 can be devoid of upper or lower strut portions, as shown in the example illustrated for frame 1102^{b} in FIG. 12B.

The length L_{S} of an angled strut 1110 of the first rung 1112 is greater than the length L_{S} of an angled strut 1110 of the second rung 1114, and/or any other rung of the frame 1102, such as the third rung 1116 and the fourth rung 1118. In some examples, the width W_{S} of an angled struts 1110 of the first rung 1112 can be larger than the width Ws of angled strut 1110 of the second rung 1114, and/or angled struts 1110 of any other rung of the frame 1102, such as the third rung 1116 and the fourth rung 1118.

In some examples, apices of the frame 1102, such as any apices defined by the first rung 1112, second rung 1114, and/or fourth rung 1118, can include arcuate regions 1176 defined between upper curved surfaces 1178 lower curved surfaces 1180, which can be similar to arcuate regions 676 described above with respect to frame 602, and may have a thickness T_{A} along a length L_{A} which is less than the thickness T_{S} of corresponding angled struts 1010 diverging therefrom. In some examples, outflow apices 1168 can include upper curved surfaces 1178 opposite to the upper ends 1146 of axial frame members 1130 extending therefrom. In some examples, the upper curved surface 1178 of an outflow apex 1168 can have a radius of curvature, which can be, in some examples, greater than 1 mm. In some examples, the radius of curvature of the upper curved surface 1178 can be in a range of 1-20 mm, 3-16 mm, or 8-14 mm. In some examples, the radius of curvature of the upper curved surface 1178 can be greater than 10 mm.

FIG. 13 shows a portion of a frame 1202 of an exemplary prosthetic valve 1200, which is similar in structure and function to frame 802 of prosthetic valve 800 disclosed above with respect to FIG. 9, with like numbers referring to like components, except that the first cell row 1252 includes two types of cells, namely elongated wide cells 1250a and elongated narrow cells 1250b, having different widths in the circumferential direction. Two axial struts 1232 are disposed between immediately adjacent commissure supports 1234 along the circumference of the first cell row 1252.

The commissure supports 1234 are attached at their upper ends 1246 to lower junctions 1266 of the first rung 1212 of angled struts, and are attached at their lower ends 1248 to lower junctions 1266 of the second rung 1214 of angled struts. The axial struts 1232 are attached at their upper ends 1246 to upper junctions 1264 of the first rung 1212 of angled struts, which are also the outflow apices 1268 of the illustrated exemplary frame 1202, and are attached at their lower ends 1248 to upper junctions 1264 of the second rung 1214 of angled struts. Thus, the commissure supports 1234 and axial struts 1232 can have substantially similar lengths (i.e., L_{C}=L_{N}).

Each elongated wide cell 1250a circumferentially extends between a commissure support 1234 and an immediately adjacent axial strut 1232, and includes three angled struts 1210 of the first rung 1212 and three angled struts 1210 of the second rung 1214 extending between the commissure support 1234 and axial strut 1232 of the cell 1250a. Thus, each elongated wide cell 1250a includes a free apex 1272 defined by angled struts 1210 of the second rung 1214. Each elongated narrow cell 1250b circumferentially extends between two immediately adjacent axial struts 1232, and includes two angled struts 1210 of the first rung 1212 and two angled struts 1210 of the second rung 1214 extending between the axial struts 1232 of the cell 1250b. Thus, the elongated wide cells 1250a and elongated narrow cells 1250b can be alternately arranged around the circumference of the first cell row 1252, such that a single elongated narrow cell 1250b is disposed between each two immediately adjacent elongated wide cells 1250a, and/or such that a single elongated wide cell 1250a is disposed between each two immediately adjacent elongated narrow cells 1250b.

Since all angled struts 1210 along the first 1212 and second 1214 rungs have identical lengths L_{S}, the elongated wide cells 1250a are wider, in the circumferential direction, than the elongated narrow cells 1250b. Each elongated wide cell 1250a can span a width of 1.5 cells 1250 of the second cell row 1254 and/or any subsequent cell row, while the elongated narrow cell 1250b can have a width similar to that of cells 1250 of the second cell row 1254 and/or any other subsequent cell row. Thus, the width of the elongated wide cell 1250a can be about 1.5 larger than the width of the elongated narrow cell 1250b.

While frames 202, 302, 402, 502, 602, 702, 802, 902, 1002, 1102 are illustrated throughout FIGS. 3A-12B to include three rows of cells, it is to be understood that any frame of an exemplary valve disclosed herein can include any other number of cells rows. For example, frame 1202 is illustrated in FIG. 13 to include, in additional to the first 1252, second 1254 and third 1256 cell rows, a fourth cell row 1258 defined between the fourth rung 1218 of angled struts and a fifth rung 1220 of angled struts, and a fifth cell row 1260 defined between the fifth rung 1220 of angled struts and a sixth rung 1222 of angled struts, wherein the inflow apices 1270 can be defined by the sixth rung 1222. It is to be understood that any other number of cell rows or rungs of angled struts is contemplated.

While apices of the frame 1202 are illustrated in FIG. 13 to be devoid of arcuate regions, it is to be understood that this is shown by way of illustration and not limitations, and that in some examples, any of the outflow apices 1268, inflow apices 1270, and/or free apices 1272, can include arcuate regions defined between upper curved surfaces lower curved surfaces, which can be similar to arcuate regions 676 described above with respect to frame 602.

In some examples, a commissure support 1234 of frame 1202 can be devoid of upper or lower strut portions, as illustrated for example in FIG. 13. In some examples, commissure supports 1234 of frame 1202 can include upper strut portions and/or lower strut portions, in a similar manner to that shown for commissure supports 334^{a}, 334^{b} or 334^{c} in FIGS. 4A-4C.

FIG. 14A shows a portion of a frame 1302 of an exemplary prosthetic valve 1300, which can be similar in structure and function to any frame disclosed herein, except that the first cell row 1352 is devoid of axial frame members, while the second cell row 1354 includes the axial struts 1332 and the commissure supports 1334. As further illustrated, the second cell row 1354 includes two types of cells, namely elongated wide cells 1350a and elongated narrow cells 1350b, and the first cell row 1352 includes differently shaped and dimensioned upper cells 1350c, wherein all three cell types 1350a, 1350b and 1350c of the first two cell rows 1352, 1354 have different widths in the circumferential direction.

Angled struts 1310 of the first rung 1312 are connected at the lower junctions 1366 of the first rung 1312 to angled struts 1310 of the second rung 1314 at the upper junctions 1364 of the second rung 1314, while the outflow apices 1368 defined by the first rung 1312 are proximal to the second rung 1314. Two axial struts 1332 are disposed between immediately adjacent commissure supports 1334 along the circumference of the second cell row 1354. The commissure supports 1334 are attached at their upper ends 1346 to the lower junctions 1366 of the first rung 1312, which are also the upper junctions 1364 of the second rung 1314, and are attached at their lower ends 1348 to upper junctions 1364 of the third rung 1316. The axial struts 1332 are attached at their upper ends 1346 to lower junctions 1366 of the second rung 1314 of angled struts, and are attached at their lower ends 1348 to upper junctions 1364 of the fourth rung 1318 of angled struts, which are also lower junctions 1366 of the third rung 1316 of angled struts. Thus, the axial struts 1332 are longer than the commissure supports 1334 (i.e., L_{N}> L_{C}).

Each elongated wide cell 1350a of the second cell row 1354 circumferentially extends between a commissure support 1334 and an immediately adjacent axial strut 1332, and includes three angled struts 1210 of the second rung 1314 and three angled struts 1310 of the third rung 1316 extending between the commissure support 1334 and axial strut 1332 of the cell 1350a. Thus, each elongated wide cell 1350a includes a free apex 1374 defined by angled struts 1310 of the third rung 1316. Each elongated narrow cell 1350b of the second cell row 1354 circumferentially extends between two immediately adjacent axial struts 1332, and includes two angled struts 1310 of the second rung 1314 and two angled struts 1310 of the fourth rung 1318 extending between the axial struts 1332 of the cell 1350b. Thus, the elongated wide cells 1350a and elongated narrow cells 1350b can be alternately arranged around the circumference of the second cell row 1354, such that a single elongated narrow cell 1350b is disposed between each two immediately adjacent elongated wide cells 1350a, and/or such that a single elongated wide cell 1350a is disposed between each two immediately adjacent elongated narrow cells 1350b.

As shown in FIG. 14A, the elongated narrow cells 1350b is devoid of struts of the third rung 1316. Thus, while each of the first rung 1312, the second rung 1314, the fourth rung 1318, and the fifth rung 1320, continuously extend around the circumference of the frame 1302, the third rung 1316 is a circumferentially discontinuous rung, being devoid of angled struts along the regions of the elongated narrow cells 1350b. Similarly, while each of the first cell row 1352, second cell row 1354, and fourth cell row 1358, continuously extend around the circumference of the frame 1302, the third cell row 1356 is a discontinuous row of cells, being devoid of cells of its own along the regions of the elongated narrow cells 1350b.

Since all angled struts 1210 along the second 1214, third 1316 and fourth 1318 rungs have identical lengths L_{S}, the elongated wide cells 1350a are wider, in the circumferential direction, than the elongated narrow cells 1350b. Each elongated wide cell 1350a can span a width of 1.5 cells 1350 of the third 1356 and fourth 1358 cell rows, while the elongated narrow cell 1350b can have a width similar to that of cells 1350 of the third 1356 and fourth 1358 cell rows. Thus, the width of the elongated wide cell 1350a can be about 1.5 larger than the width of the elongated narrow cell 1350b.

The length L_{S} of an angled strut 1310 of the first rung 1312 is greater than the length L_{S} of an angled strut 1310 of any of the subsequent rungs, such as the second rung 1314, the third rung 1316, the fourth rung 1318, and the fifth rung 1320. Each upper cell 1350c of the first cell row 1352 is defined by two angled struts 1310 of the first rung 1312 and four angled struts 1310 of the second rung 1314. Thus, each upper cell 1350c includes a free apex 1372 defined by angled struts 1310 of the second rung 1314. Each upper cell 1350c of the first cell row 1352 spans the width of two cells 1350 of the third 1356 and fourth 1358 cell rows. Thus, each upper cell 1350c of the first cell row 1352 is wider than any of the cells 1350a or 1350b of the second cell row 1354. In some examples, the width W_{S} of an angled struts 1310 of the first rung 1312 can be larger than the width Ws of angled struts 1310 of any of the subsequent rungs, such as the second rung 1314, the third rung 1316, the fourth rung 1318, and the fifth rung 1320.

In some examples, apices of the frame 1302, such as any of the outflow apices 1368, inflow apices 1370, free apices 1372 defined by the second rung 1314, and/or free apices 1374 defined by the third rung 1316, can include arcuate regions 1376 defined between upper curved surfaces 1378 lower curved surfaces 1380, which can be similar to arcuate regions 676 described above with respect to frame 602.

In some examples, a commissure support 1334 of frame 1302 can be devoid of upper or lower strut portions, as illustrated for example in FIG. 14A. In some examples, commissure supports 1334 of frame 1302 can include upper strut portions and/or lower strut portions, in a similar manner to that shown for commissure supports 334^{a}, 334^{b} or 334^{c} in FIGS. 4A-4C.

FIG. 14A shows an exemplary frame 1302^{a}, which is similar to any example described herein for frame 1302, except that the fourth cell row 1358 is the lowermost cell row, and the fifth rung 1320 is the lowermost rung of angled struts, defining the inflow apices 1370. The prosthetic valve 1300 includes a valvular structure 184 coupled to the frame 1302. FIG. 14A illustrates relative exemplary positions of a leaflet 186 and the frame 1302^{a} after mounting the leaflet 186 to the frame 1302^{a} (the outer perimeter of the leaflet is shown in dashed lines).

As shown in FIG. 14A, the free edge 124 of the leaflet 186 can extend across two upper cells 1350c of the first cell row 1352. Each tab 196 of the leaflet 186 can be paired with an adjacent tab 196 of an adjacent leaflet 186 to form a commissure 188 of the valvular structure 184 that is inserted into a corresponding commissure window 1336. The cusp edge 126 of the leaflet 186 can extend along and be attached to at least six angled struts 1310, such as two angled struts 1310 of each of the third rung 1316, fourth rung 1318, and fifth rung 1320. The apex 128 of leaflet can be located at the inflow end 1306 of the frame 1302^{a}. In the example illustrated in FIG. 14A, the apex 128 of the leaflet is not connected to any strut of the frame 1302^{a}. The apex 128 of the leaflet can be located upstream of an upper junction 1364 of the fifth rung 1320 where the elongated narrow cell 1350b is connected to the fifth rung 1320 of angles struts.

FIG. 14B shows an exemplary frame 1302^{b}, which is similar to any example described herein for frame 1302, except that the frame 1302^{b} further comprises a fifth cell row 1360 defined by angled struts 1310 of the fifth rung 1320 and a sixth rung 1322, with the sixth rung 1322 defining the inflow apices 1370. FIG. 14B further illustrates relative exemplary positions of a leaflet 186 and the frame 1302^{b} after mounting the leaflet 186 to the frame 1302^{b} (the outer perimeter of the leaflet is shown in dashed lines).

As shown in FIG. 14B, the free edge 124 of the leaflet 186 can extend across two upper cells 1350c of the first cell row 1352. Each tab 196 of the leaflet 186 can be paired with an adjacent tab 196 of an adjacent leaflet 186 to form a commissure 188 of the valvular structure 184 that is inserted into a corresponding commissure window 1336. The cusp edge 126 of the leaflet 186 can extend along and be attached to at least eight angled struts 1310, such as two angled struts 1310 of each of the third rung 1316, fourth rung 1318, fifth rung 1320, and sixth rung 1322. The apex 128 of the leaflet can align with an inflow apex 1370 of the frame 1302^{b}.

FIG. 15 shows a portion of a frame 1402 of an exemplary prosthetic valve 1400, which can be similar in structure and function to frame 1002 of prosthetic valve 1000 disclosed above with respect to FIGS. 11A-11B, except that each cell 1450 of the first cell row 1452 and the second cell row 1454 has a width, in the circumferential direction, that spans the width of two cells of any subsequent cell row, such as the third cell row 1356, fourth cell row 1358, and/or fifth cell row 1460. The axial frame members 1430 of the frame 1402, including the axial struts 1432 and commissure supports 1434, are attached at their upper ends 1446 to lower junctions 1466 of the first rung 1412 of angled struts, and are attached at their lower ends 1448 to lower junctions 1466 of the second rung 1414 of angled struts, which are also upper junctions 1464 of the third rung 1416 of angled struts. Thus, the commissure supports 1434 and the axial struts 1432 can have substantially similar lengths (i.e., L_{C}=L_{N}).

Each cell 1450 of the first cell row 1452 includes two angled struts 1410 of the first rung 1412 and two angled struts 1410 of the second rung 1414 extending between immediately adjacent axial frame members 1130. Thus, each cell 1450 of the first cell row 1452 includes a free apex 1472 defined by angled struts 1410 of the second rung 1414. Each cell 1450 of the second cell row 1454 is defined by two angled struts 1410 of the second rung 1412 and four angled struts 1410 of the third rung 1414. Thus, each cell 1450 of the second cell row 1454 includes a free apex 1474 defined by angled struts 1410 of the third rung 1416. The inclusion of the angled struts of the second rung, inside a space defined between the first rung and the third rung, can advantageously prevent the leaflets from extending outwardly through cell of the frame through cell openings that may be otherwise too large.

In some examples, each of the first cell row 1452 and the second cell row 1454 includes six cells 1150, while any subsequent cell row, such as the third cell row 1456, fourth cells row 1458, and/or fifth cell row 1460, can include twelve cells 1450. The frame 1402 can include a single axial strut 1432 disposed between immediately adjacent commissure support 1434.

The length L_{S} of an angled strut 1410 of the first rung 1412 is equal to the length L_{S} of an angled strut 1410 of the second rung 1414, and is greater than the length L_{S} of an angled strut 1110 of subsequent rungs, such as any of the third rung 1416, fourth rung 1418, fifth rung 1420, and/or sixth rung 1422. In some examples, the width W_{S} of an angled strut 1410 of the first rung 1412 or second rung 1414 can be larger than the width Ws of angled struts 1410 of subsequent rungs, such as any of the third rung 1416, fourth rung 1418, fifth rung 1420, and/or sixth rung 1422.

In some examples, apices of the frame 1402, such as any of the outflow apices 1468, inflow apices 1470, free apices 1472 defined by the second rung 1414, and/or free apices 1474 defined by the third rung 1416, can include arcuate regions 1476 defined between upper curved surfaces 1478 lower curved surfaces 1480, which can be similar to arcuate regions 676 described above with respect to frame 602.

In some examples, a commissure support 1434 of frame 1402 can be devoid of upper or lower strut portions, as illustrated for example in FIG. 15. In some examples, commissure supports 1434 of frame 1402 can include upper strut portions and/or lower strut portions, in a similar manner to that shown for commissure supports 334^{a}, 334^{b} or 334^{c} in FIGS. 4A-4C.

FIG. 15 further illustrates relative exemplary positions of a leaflet 186 and the frame 1402 after mounting the leaflet 186 to the frame 1402 (the outer perimeter of the leaflet is shown in dashed lines). As shown, the free edge 124 of the leaflet 186 can extend across two cells 1450 of the first cell row 1412. Each tab 196 of the leaflet 186 can be paired with an adjacent tab 196 of an adjacent leaflet 186 to form a commissure 188 of the valvular structure 184 that is inserted into a corresponding commissure window 1436. The cusp edge 126 of the leaflet 186 can extend along and be attached to at least eight angled struts 1410, such as two angled struts 1410 of each of the third rung 1416, fourth rung 1418, fifth rung 1420, and sixth rung 1422. The apex 128 of the leaflet can align with an inflow apex 1470 of the frame 1402. It is to be understood that in some examples, the frame 1402 can include more or less cell rows and/or more or less rungs of angled struts, and that other attachment configurations of a leaflet 186 to the frame 1402 are contemplated, including such that the apex 128 of the leaflet can remain unconnected to any strut of the frame 1402.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:

a frame movable between a radially compressed state and a radially expanded state, the frame extending between an inflow end and an outflow end and comprising:

a first rung of angled struts interconnected at upper junctions and lower junctions of the first rung;

a second rung of angled struts interconnected at upper junctions and lower junctions of the second rung, wherein the second rung is distal to the first rung;

a third rung of angled struts interconnected at upper junctions and lower junctions of the first rung, wherein the third rung is distal to the second rung;

a first cell row comprising:

a plurality of axial frame members, each axial frame member connected at an upper end thereof to the first rung, and at a lower end thereof to the second rung; and

a plurality of cells, wherein at least one cell of the first cell row is defined between a plurality of angled struts of the first rung, two of the axial frame members, and at least three angled struts of the second rung; and

a second cell row comprising a plurality of cells, each cell of the second cell row defined between two angled struts of the second rung and two angled struts of the third rung;

wherein at least some of the cells of the first cell row are wider, in the circumferential direction, than the cells of the second cell row.

Example 2. The prosthetic valve of any example herein, particularly of example 1, wherein the upper junctions of the first rung are outflow apices of the frame.

Example 3. The prosthetic valve of any example herein, particularly of any one of examples 1 or 2, wherein the axial frame members are parallel to each other.

Example 4. The prosthetic valve of any example herein, particularly of any one of examples 1 to 3, wherein the axial frame members are parallel to a central axis of the frame.

Example 5. The prosthetic valve of any example herein, particularly of any one of examples 1 to 4, wherein the second cell row is closer to the inflow end than the first cell row.

Example 6. The prosthetic valve of any example herein, particularly of any one of examples 1 to 5, wherein the angled struts of each of the first rung, the second rung, and the third rung, are circumferentially arranged in a zig-zagged pattern.

Example 7. The prosthetic valve of any example herein, particularly of any one of examples 1 to 6, wherein the axial frame members comprise a plurality of axial struts and a plurality of commissure supports.

Example 8. The prosthetic valve of any example herein, particularly of example 7, wherein the commissure support is configured to support a commissure of a valvular structure that can be mounted in the frame.

Example 9. The prosthetic valve of any example herein, particularly of any one of examples 7 or 8, wherein each of the plurality of commissure supports comprises a commissure window.

Example 10. The prosthetic valve of any example herein, particularly of example 9, wherein the commissure window comprises a window opening between two window struts of the commissure window.

Example 11. The prosthetic valve of any example herein, particularly of any one of examples 7 to 10, wherein at least one cell of the first cell row spans a width of one and a half cells of the second cell row.

Example 12. The prosthetic valve of any example herein, particularly of example 11, wherein the second rung comprises three angled struts extending between immediately adjacent axial frame members along the first cell row.

Example 13. The prosthetic valve of any example herein, particularly of example 12, wherein the first rung comprises three angled struts extending between immediately adjacent axial frame members along the first cell row.

Example 14. The prosthetic valve of any example herein, particularly of any one of examples 12 or 13, wherein the commissure supports are attached at their upper ends to the upper junctions of the first rung, and are attached at their lower ends to lower junctions of the second rung.

Example 15. The prosthetic valve of any example herein, particularly of example 14, wherein the axial struts are attached at their upper ends to the lower junctions of the first rung, and are attached at their lower ends to upper junctions of the second rung.

Example 16. The prosthetic valve of any example herein, particularly of any one of examples 14 or 15, wherein the commissure supports are longer than the axial struts.

Example 17. The prosthetic valve of any example herein, particularly of any one of examples 12 or 13, wherein the commissure supports are attached at their upper ends to the lower junctions of the first rung, and are attached at their lower ends to upper junctions of the second rung.

Example 18. The prosthetic valve of any example herein, particularly of examples 17, wherein the axial struts are attached at their upper ends to the upper junctions of the first rung, and are attached at their lower ends to lower junctions of the second rung.

Example 19. The prosthetic valve of any example herein, particularly of any one of examples 17 or 18, wherein the axial struts are longer than the commissure supports.

Example 20. The prosthetic valve of any example herein, particularly of any one of examples 12 or 13, wherein the commissure supports are attached at their upper ends to the lower junctions of the first rung, and are attached at their lower ends to lower junctions of the second rung.

Example 21. The prosthetic valve of any example herein, particularly of example 20, wherein the axial struts are attached at their upper ends to the upper junctions of the first rung, and are attached at their lower ends to upper junctions of the second rung.

Example 22. The prosthetic valve of any example herein, particularly of any one of examples 12 or 13, wherein the commissure supports are attached at their upper ends to the upper junctions of the first rung, and are attached at their lower ends to upper junctions of the second rung.

Example 23. The prosthetic valve of any example herein, particularly of example 22, wherein the axial struts are attached at their upper ends to the lower junctions of the first rung, and are attached at their lower ends to lower junctions of the second rung.

Example 24. The prosthetic valve of any example herein, particularly of any one of examples 20 to 23, wherein the axial struts and the commissure supports have equal lengths.

Example 25. The prosthetic valve of any example herein, particularly of example 12, wherein the first rung comprises two angled struts extending between immediately adjacent axial frame members along the first cell row.

Example 26. The prosthetic valve of any example herein, particularly of example 25, wherein the commissure supports are attached at their upper ends to the lower junctions of the first rung, and are attached at their lower ends to lower junctions of the second rung.

Example 27. The prosthetic valve of any example herein, particularly of example 26, wherein the axial struts are attached at their upper ends to the lower junctions of the first rung, and are attached at their lower ends to upper junctions of the second rung.

Example 28. The prosthetic valve of any example herein, particularly of any one of examples 25 to 27, wherein the commissure supports are longer than the axial struts.

Example 29. The prosthetic valve of any example herein, particularly of any one of examples 25 to 28, wherein each upper junction of the first rung is circumferentially aligned with a lower junction of the second rung.

Example 30. The prosthetic valve of any example herein, particularly of any one of examples 25 to 29, wherein each angled strut of the first rung, extending between the upper end of the corresponding commissure support and the upper junction of the first rung, has a greater strut length than that of the angled strut of the first rung extending between the same upper junction of the first rung and the upper end of the corresponding axial strut.

Example 31. The prosthetic valve of any example herein, particularly of any one of examples 7 to 10, wherein each cell of the first cell row spans a width of two cells of the second cell row.

Example 32. The prosthetic valve of any example herein, particularly of example 31, wherein the second rung comprises four angled struts extending between immediately adjacent axial frame members along the first cell row.

Example 33. The prosthetic valve of any example herein, particularly of example 32, wherein the first rung comprises two angled struts extending between immediately adjacent axial frame members along the first cell row.

Example 34. The prosthetic valve of any example herein, particularly of example 33, wherein the commissure supports are attached at their upper ends to the upper junctions of the first rung, and are attached at their lower ends to the upper junctions of the second rung.

Example 35. The prosthetic valve of any example herein, particularly of example 34, wherein the axial struts are attached at their upper ends to the upper junctions of the first rung, and are attached at their lower ends to the upper junctions of the second rung.

Example 36. The prosthetic valve of any example herein, particularly of any one of examples 31 to 35, wherein the axial struts and the commissure supports have equal lengths.

Example 37. The prosthetic valve of any example herein, particularly of any one of examples 25-28 or 33-35, wherein each angled strut of the first rung has a strut length greater than a strut length of any angled strut of the second rung.

Example 38. The prosthetic valve of any example herein, particularly of any one of examples 25-28 or 33-35, wherein each angled strut of the first rung has a strut width greater than a strut width of any angled strut of the second rung.

Example 39. The prosthetic valve of any example herein, particularly of any one of examples 12 to 28, wherein the first cell row comprises six cells and the second cell row comprises nine cells.

Example 40. The prosthetic valve of any example herein, particularly of any one of examples 31 to 36, wherein the first cell row comprises six cells and the second cell row comprises twelve cells.

Example 41. The prosthetic valve of any example herein, particularly of any one of examples 12 to 40, wherein a single one of the axial struts is disposed between immediately adjacent commissure supports.

Example 42. The prosthetic valve of any example herein, particularly of any one of examples 12 to 41, wherein a single one of the commissure supports is disposed between immediately adjacent axial struts.

Example 43. The prosthetic valve of any example herein, particularly of any one of examples 12 to 42, wherein the plurality of axial struts comprises three axial struts, and wherein the plurality of commissure supports comprises three commissure supports.

Example 44. The prosthetic valve of any example herein, particularly of any one of examples 12 to 43, wherein the axial struts and the commissure supports are alternately arranged around the circumference of the first cell row.

Example 45. The prosthetic valve of any example herein, particularly of any one of examples 12 to 44, wherein each cell of the first cell row comprises a free apex defined by the second rung.

Example 46. The prosthetic valve of any example herein, particularly of example 45, wherein each free apex comprises an arcuate region defined between an upper curved surface and a lower curved surface thereof.

Example 47. The prosthetic valve of any example herein, particularly of example 46, wherein a width of the arcuate region of each free apex is less than a width of an angled strut extending therefrom.

Example 48. The prosthetic valve of any example herein, particularly of example 11, wherein the cells of the first cell row comprise elongated wide cells and elongated narrow cells, wherein the elongated wide cells are wider, in the circumferential direction, than the elongated narrow cells.

Example 49. The prosthetic valve of any example herein, particularly of example 48, wherein each elongated wide cell spans a width of one and a half cells of the second cell row.

Example 50. The prosthetic valve of any example herein, particularly of any one of examples 48 or 49, wherein each elongated narrow cell and each cell of the second cell row have equal widths in the circumferential direction.

Example 51. The prosthetic valve of any example herein, particularly of any one of examples 48 to 50, wherein each elongated wide cell comprises one of the plurality of commissure supports, one of the plurality of axial struts, three angled struts of the first rung extending between the corresponding commissure support and the axial strut, and three angled struts of the second rung extending between the corresponding commissure support and the axial strut.

Example 52. The prosthetic valve of any example herein, particularly of any one of examples 48 to 51, wherein each elongated narrow cell comprises two of the plurality of axial struts, two angled struts of the first rung extending between the corresponding axial struts, and two angled struts of the second rung extending between the corresponding axial struts.

Example 53. The prosthetic valve of any example herein, particularly of any one of examples 48 to 52, wherein two axial struts are disposed between immediately adjacent commissure supports.

Example 54. The prosthetic valve of any example herein, particularly of any one of examples 48 to 53, wherein the elongated wide cells and the elongated narrow cells are alternately arranged around the circumference of the first cell row.

Example 55. The prosthetic valve of any example herein, particularly of any one of examples 48 to 54, wherein each elongated wide cell comprises a free apex defined by the second rung.

Example 56. The prosthetic valve of any example herein, particularly of example 55, wherein each free apex comprises an arcuate region defined between an upper curved surface and a lower curved surface thereof.

Example 57. The prosthetic valve of any example herein, particularly of example 56, wherein a width of the arcuate region of each free apex is less than a width of an angled strut extending therefrom.

Example 58. The prosthetic valve of any example herein, particularly of any one of examples 12 to 57, wherein each upper junction of the first rung comprises an arcuate region defined between an upper curved surface and a lower curved surface thereof.

Example 59. The prosthetic valve of any example herein, particularly of example 58, wherein a width of the arcuate region of each upper junction of the first rung is less than a width of an angled strut extending therefrom.

Example 60. The prosthetic valve of any example herein, particularly of any one of examples 7 to 59, each commissure support further comprises an upper strut portion extending between the commissure window and the upper end of the commissure support.

Example 61. The prosthetic valve of any example herein, particularly of example 60, wherein each commissure support further comprises a lower strut portion extending between the commissure window and the lower end of the commissure support.

Example 62. The prosthetic valve of any example herein, particularly of any one of examples 7 to 59, each commissure support further comprises a lower strut portion extending between the commissure window and the lower end of the commissure support.

Example 63. The prosthetic valve of any example herein, particularly of any one of examples 7 to 62, further comprising a fourth rung of angled struts distal to the third rung.

Example 64. The prosthetic valve of any example herein, particularly of any one of examples 7 to 63, further comprising a third cell row comprising a plurality of cells, each cell of the third cell row defined between two angled struts of the third rung and two angled struts of the fourth rung.

Example 65. The prosthetic valve of any example herein, particularly of any one of examples 1-64, further comprising a valvular structure mounted inside the frame and comprising a plurality of leaflets, each leaflet comprising a free edge closer to the outflow end of the frame, and a cusp edge along which the leaflet is coupled to the frame.

Example 66. The prosthetic valve of any example herein, particularly of example 65, wherein the cusp edge is coupled to two angled struts of the second rung, two angled struts of the third rungs, and two angled struts of the third rung.

Example 67. The prosthetic valve of any example herein, particularly of any one of examples 65 or 66, wherein an apex of the cusp edge is located at the inflow end of the frame.

Example 68. The prosthetic valve of any example herein, particularly of example 67, wherein the apex of the cusp edge is not connected to the frame.

Example 69. The prosthetic valve of any example herein, particularly of example 67, wherein the apex of the cusp edge is aligned with an inflow apex of the frame.

Example 70. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a plurality of axial frame members extending between upper ends and lower ends thereof, the axial frame members comprising:

a plurality of commissure supports, each commissure support comprising a commissure window and an upper strut portion extending between the commissure window and the upper end, wherein each commissure support is attached at its upper end to an upper junction of the first rung, and is attached at its lower end to a lower junction of the second rung; and

a plurality of axial struts, wherein each axial strut is attached at its upper end to a lower junction of the first rung, and is attached at its lower end to an upper junction of the second rung;

wherein the first rung comprises three angled struts extending between immediately adjacent axial frame members, and the second rung comprises three angled struts extending between immediately adjacent axial frame members.

Example 71. The prosthetic valve of any example herein, particularly of example 70, wherein each commissure support further comprises a lower strut portion extending between the commissure window and the lower end of the commissure support.

Example 72. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a plurality of axial frame members extending between upper ends and lower ends thereof, the axial frame members comprising:

a plurality of commissure supports, each commissure support comprising a commissure window and at least one strut portion axially extending from the commissure window, wherein each commissure support is attached at its upper end to a lower junction of the first rung, and is attached at its lower end to an upper junction of the second rung; and

a plurality of axial struts, wherein each axial strut is attached at its upper end to an upper junction of the first rung, and is attached at its lower end to a lower junction of the second rung;

wherein the first rung comprises three angled struts extending between immediately adjacent axial frame members, and the second rung comprises three angled struts extending between immediately adjacent axial frame members.

Example 73. The prosthetic valve of any example herein, particularly of example 72, wherein the at least one strut portion comprises an upper strut portion extending between the commissure window and the upper end of the commissure support.

Example 74. The prosthetic valve of any example herein, particularly of example 73, wherein the at least one strut portion further comprises a lower strut portion extending between the commissure window and the lower end of the commissure support.

Example 75. The prosthetic valve of any example herein, particularly of example 72, wherein the at least one strut portion comprises a lower strut portion extending between the commissure window and the lower end of the commissure support.

Example 76. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a plurality of axial frame members extending between upper ends and lower ends thereof, the axial frame members comprising:

a plurality of commissure supports, each commissure support comprising a commissure window and at least one strut portion axially extending from the commissure window; and

a plurality of axial struts, wherein each axial strut has a strut length equal to a strut length of any of the commissure supports;

wherein the first rung comprises three angled struts extending between immediately adjacent axial frame members, and the second rung comprises three angled struts extending between immediately adjacent axial frame members.

Example 77. The prosthetic valve of any example herein, particularly of example 76, wherein each commissure support is attached at its upper end to a lower junction of the first rung, and is attached at its lower end to a lower junction of the second rung.

Example 78. The prosthetic valve of any example herein, particularly of any one of examples 76 or 77, wherein each axial strut is attached at its upper end to an upper junction of the first rung, and is attached at its lower end to an upper junction of the second rung.

Example 79. The prosthetic valve of any example herein, particularly of example 76, wherein each commissure support is attached at its upper end to an upper junction of the first rung, and is attached at its lower end to an upper junction of the second rung.

Example 80. The prosthetic valve of any example herein, particularly of any one of examples 76 or 79, wherein each axial strut is attached at its upper end to a lower junction of the first rung, and is attached at its lower end to a lower junction of the second rung.

Example 81. The prosthetic valve of any example herein, particularly of any one of examples 76 to 80, wherein the at least one strut portion comprises an upper strut portion extending between the commissure window and the upper end of the commissure support.

Example 82. The prosthetic valve of any example herein, particularly of example 81, wherein the at least one strut portion further comprises a lower strut portion extending between the commissure window and the lower end of the commissure support.

Example 83. The prosthetic valve of any example herein, particularly of any one of examples 76 to 80, wherein the at least one strut portion comprises a lower strut portion extending between the commissure window and the lower end of the commissure support.

Example 84. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a plurality of axial frame members, each axial frame member connected at an upper end thereof to the first rung, and at a lower end thereof to the second rung;

wherein the first rung comprises two angled struts extending between immediately adjacent axial frame members; and

wherein the second rung comprises more than two angled struts extending between immediately adjacent axial frame members.

Example 85. The prosthetic valve of any example herein, particularly of example 84, wherein the axial frame members comprise a plurality of axial struts and a plurality of commissure supports, each commissure support comprising a commissure window.

Example 86. The prosthetic valve of any example herein, particularly of example 85, wherein the second rung comprises three angled struts extending between immediately adjacent axial frame members.

Example 87. The prosthetic valve of any example herein, particularly of example 86, wherein each commissure support is attached at its upper end to a lower junction of the first rung, and is attached at its lower end to a lower junction of the second rung.

Example 88. The prosthetic valve of any example herein, particularly of any one of examples 86 or 87, wherein each axial strut is attached at its upper end to a lower junction of the first rung, and is attached at its lower end to an upper junction of the second rung.

Example 89. The prosthetic valve of any example herein, particularly of any one of examples 86 to 88, wherein each upper junction of the first rung comprises an arcuate region defined between an upper curved surface and a lower curved surface thereof.

Example 90. The prosthetic valve of any example herein, particularly of example 89, wherein a width of the arcuate region of each upper junction of the first rung is less than a width of an angled strut extending therefrom.

Example 91. The prosthetic valve of any example herein, particularly of any one of examples 86 to 90, wherein each upper junction of the first rung is circumferentially aligned with a lower junction of the second rung.

Example 92. The prosthetic valve of any example herein, particularly of any one of examples 86 to 91, wherein each angled strut of the first rung, extending between the upper end of the corresponding commissure support and the upper junction of the first rung, has a greater strut length than that of the angled strut of the first rung extending between the same upper junction of the first rung and the upper end of the corresponding axial strut.

Example 93. The prosthetic valve of any example herein, particularly of example 85, wherein the second rung comprises four angled struts extending between immediately adjacent axial frame members.

Example 94. The prosthetic valve of any example herein, particularly of example 93, wherein each commissure support is attached at its upper end to an upper junction of the first rung, and is attached at its lower end to an upper junction of the second rung.

Example 95. The prosthetic valve of any example herein, particularly of any one of examples 93 or 94, wherein each axial strut is attached at its upper end to an upper junction of the first rung, and is attached at its lower end to an upper junction of the second rung.

Example 96. The prosthetic valve of any example herein, particularly of any one of examples 93 to 95, wherein each upper junction of the first rung comprises an arcuate region defining an upper curved surface opposite to the axial frame member attached to the upper junction of the first rung.

Examples 97. The prosthetic valve of any example herein, particularly of any one of examples 85 to 96, wherein each commissure support further comprises an upper strut portion extending between the commissure window and the upper end of the commissure support.

Example 98. The prosthetic valve of any example herein, particularly of example 97, wherein each commissure support further comprises a lower strut portion extending between the commissure window and the lower end of the commissure support.

Example 99. The prosthetic valve of any example herein, particularly of any one of examples 85 to 96, wherein each commissure support further comprises a lower strut portion extending between the commissure window and the lower end of the commissure support.

Example 100. The prosthetic valve of any example herein, particularly of any one of examples 84 to 99, further comprising free apices defined by the second rung between immediately adjacent axial frame members.

Example 101. The prosthetic valve of any example herein, particularly of example 100, wherein each free apex comprises an arcuate region defined between an upper curved surface and a lower curved surface thereof.

Example 102. The prosthetic valve of any example herein, particularly of example 101, wherein a width of the arcuate region of each free apex is less than a width of an angled strut extending therefrom.

Example 103. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a first cell row comprising:

a plurality of commissure supports, wherein each commissure support is attached at its upper end to a lower junction of the first rung, and is attached at its lower end to a lower junction of the second rung;

a plurality of axial struts, wherein each axial strut is attached at its upper end to an upper junction of the first rung, and is attached at its lower end to an upper junction of the second rung;

a plurality of elongated wide cells, wherein each elongated wide cell comprises one of the plurality of commissure supports, one of the plurality of axial struts, three angled struts of the first rung extending between the corresponding commissure support and the axial strut, and three angled struts of the second rung extending between the corresponding commissure support and the axial strut; and

a plurality of elongated narrow cells, wherein each elongated narrow cell comprises two of the plurality of axial struts, two angled struts of the first rung extending between the corresponding axial struts, and two angled struts of the second rung extending between the corresponding axial struts.

Example 104. The prosthetic valve of any example herein, particularly of example 103, wherein two axial struts are disposed between immediately adjacent commissure supports.

Example 105. The prosthetic valve of any example herein, particularly of any one of examples 103 or 104, wherein the elongated wide cells and the elongated narrow cells are alternately arranged around the circumference of the first cell row.

Example 106. The prosthetic valve of any example herein, particularly of any one of examples 103 to 105, wherein each elongated wide cell comprises a free apex defined by the second rung.

Example 107. The prosthetic valve of any example herein, particularly of example 106, wherein each free apex comprises an arcuate region defined between an upper curved surface and a lower curved surface thereof.

Example 108. The prosthetic valve of any example herein, particularly of any one of examples 107, wherein a width of the arcuate region of each free apex is less than a width of an angled strut extending therefrom.

Example 109. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a third rung of angled struts interconnected at upper junctions and lower junctions of the third rung, wherein the third rung is distal to the second rung;

a fourth rung of angled struts interconnected at upper junctions and lower junctions of the fourth rung, wherein the fourth rung is distal to the third rung;

a first cell row comprising a plurality of upper cells, wherein each upper cell is defined between two angled struts of the first rung and four angles struts of the second rung;

a second cell row comprising:

a plurality of commissure supports, wherein each commissure support is attached at its upper end to an upper junction of the second rung, and is attached at its lower end to an upper junction of the third rung;

a plurality of axial struts, wherein each axial strut is attached at its upper end to a lower junction of the second rung, and is attached at its lower end to an upper junction of the fourth rung;

a plurality of elongated wide cells, wherein each elongated wide cell comprises one of the plurality of commissure supports, one of the plurality of axial struts, three angled struts of the second rung extending between the corresponding commissure support and the axial strut, and three angled struts of the third rung extending between the corresponding commissure support and the axial strut; and

a plurality of elongated narrow cells, wherein each elongated narrow cell comprises two of the plurality of axial struts, two angled struts of the second rung extending between the corresponding axial struts, and two angled struts of the fourth rung extending between the corresponding axial struts.

Example 110. The prosthetic valve of any example herein, particularly of example 109, wherein the third rung is discontinuous in the circumferential direction, being devoid of angled struts at regions of the elongated narrow cells.

Example 111. The prosthetic valve of any example herein, particularly of any one of examples 109 or 110, wherein each upper cell is wider, in the circumferential direction, than any of the elongated wide cells and the elongated narrow cells.

Example 112. The prosthetic valve of any example herein, particularly of any one of examples 109 to 111, wherein the frame further comprises a third cell row comprising a plurality of cells, each cell of the third cell row comprising two angled struts of the third rung and two angled struts of the fourth rung.

Example 113. The prosthetic valve of any example herein, particularly of any one of examples 112, wherein the third cell row is discontinuous in the circumferential direction, being devoid of cells at regions of the elongated narrow cells.

Example 114. The prosthetic valve of any example herein, particularly of any one of examples 112 or 113, wherein each upper cell spans a width of two cells of the third cell row.

Example 115. The prosthetic valve of any example herein, particularly of any one of examples 109 to 114, wherein two axial struts are disposed between immediately adjacent commissure supports.

Example 116. The prosthetic valve of any example herein, particularly of any one of examples 109 to 115, wherein the elongated wide cells and the elongated narrow cells are alternately arranged around the circumference of the second cell row.

Example 117. The prosthetic valve of any example herein, particularly of any one of examples 109 to 116, wherein each elongated wide cell comprises a free apex defined by the second rung, and a free apex defined by the third rung.

Example 118. The prosthetic valve of any example herein, particularly of example 117, wherein each free apex of the second rung and the third rung comprises an arcuate region defined between an upper curved surface and a lower curved surface thereof.

Example 119. The prosthetic valve of any example herein, particularly of example 118, wherein a width of the arcuate region of each free apex is less than a width of the angled strut extending therefrom.

Example 120. The prosthetic valve of any example herein, particularly of any one of examples 109 to 119, wherein a strut length of each angled strut of the first rung is greater than a strut length of any of the angled struts of the second rung, the third rung, and the fourth rung.

Example 121. The prosthetic valve of any example herein, particularly of any one of examples 109 to 120, wherein all angled struts of the second rung, the third rung, and the fourth rung, have equal strut lengths.

Example 122. The prosthetic valve of any example herein, particularly of any one of examples 109 to 121, wherein each angled strut of the first rung has a strut width greater than a strut width of any angled strut of the second rung, the third rung, and the fourth rung.

Example 123. The prosthetic valve of any example herein, particularly of any one of examples 109 to 122, wherein the upper junctions of the first rung are outflow apices of the frame, and wherein each outflow apex comprises an arcuate region defined between an upper curved surface and a lower curved surface thereof.

Example 124. The prosthetic valve of any example herein, particularly of example 118, wherein a width of the arcuate region of each outflow apex is less than a width of the angled strut extending therefrom.

Example 125. The prosthetic valve of any example herein, particularly of any one of examples 109 to 124, wherein the frame further comprises a fifth rung comprising a plurality of angled struts, and a fourth cell row comprising a plurality of cells, each cell of the fourth cell row comprising two angled struts of the fourth rung and two angled struts of the fifth rung.

Example 126. The prosthetic valve of any example herein, particularly of example 125, wherein the frame further comprises a sixth rung comprising a plurality of angled struts, and a fifth cell row comprising a plurality of cells, each cell of the fifth cell row comprising two angled struts of the fifth rung and two angled struts of the sixth rung.

Example 127. The prosthetic valve of any example herein, particularly of any one of examples 109 to 126, further comprising a valvular structure mounted inside the frame and comprising a plurality of leaflets, each leaflet comprising a free edge closer to the first rung of the frame, and a cusp edge along which the leaflet is coupled to the frame.

Example 128. The prosthetic valve of any example herein, particularly of example 127, wherein an apex of the cusp edge is located at an inflow end of the frame.

Example 129. The prosthetic valve of any example herein, particularly of example 128, wherein the apex of the cusp edge is not connected to the frame.

Example 130. The prosthetic valve of any example herein, particularly of example 128, wherein the apex of the cusp edge is aligned with an inflow apex of the frame.

Example 131. The prosthetic valve of any example herein, particularly of any one of examples 127 to 130, wherein the plurality of leaflets comprises three leaflets.

Example 132. A prosthetic valve comprising:

a frame movable between a radially compressed state and a radially expanded state, the frame comprising:

a first rung of angled struts interconnected at upper junctions and lower junctions of the first rung;

a second rung of angled struts interconnected at upper junctions and lower junctions of the second rung, wherein the second rung is distal to the first rung;

a third rung of angled struts interconnected at upper junctions and lower junctions of the third rung, wherein the third rung is distal to the second rung;

a fourth rung of angled struts interconnected at upper junctions and lower junctions of the fourth rung, wherein the fourth rung is distal to the third rung;

a first cell row comprising:

a plurality of axial frame members, each axial frame member connected at an upper end thereof to a lower junction of the first rung, and at a lower end thereof to a lower junctions of the second rung; and

a plurality of cells, each cell of the first cell row comprises two immediately adjacent axial frame members, two elongated struts of the first rung extending between the two immediately adjacent axial frame members; and two elongated struts of the second rung extending between the two immediately adjacent axial frame members;

a second cell row comprising a plurality of cells, each cell of the second cell row defined between two angled struts of the second rung and four angled struts of the third rung; and

a third cell row comprising a plurality of cells, each cell of the third cell row defined between two angled struts of the third rung and two angled struts of the fourth rung.

Example 133. The prosthetic valve of any example herein, particularly of example 132, wherein each cell of the first cell row spans a width, in the circumferential direction, of two cells of the third cell row.

Example 134. The prosthetic valve of any example herein, particularly of any one of examples 132 or 133, wherein each cell of the first cell row spans a width, in the circumferential direction, of two cells of the third cell row.

Example 135. The prosthetic valve of any example herein, particularly of any one of examples 132 to 134, wherein the upper junctions of the first rung are outflow apices of the frame, and wherein each outflow apex comprises an arcuate region defined between an upper curved surface and a lower curved surface thereof.

Example 136. The prosthetic valve of any example herein, particularly of example 135, wherein a width of the arcuate region of each outflow apex is less than a width of the angled strut extending therefrom.

Example 137. The prosthetic valve of any example herein, particularly of any one of examples 132 to 136, wherein each cell of the first cell row further comprises a free apex defined by the second rung.

Example 138. The prosthetic valve of any example herein, particularly of example 137, wherein each free apex comprises an arcuate region defined between an upper curved surface and a lower curved surface thereof.

Example 139. The prosthetic valve of any example herein, particularly of example 138, wherein a width of the arcuate region of each free apex is less than a width of an angled strut extending therefrom.

Example 140. The prosthetic valve of any example herein, particularly of any one of examples 132 to 139, wherein a strut length of each angled strut of the first rung and the second rung is greater than a strut length of any of the angled struts of the third rung and the fourth rung.

Example 141. The prosthetic valve of any example herein, particularly of any one of examples 132 to 140, wherein all angled struts of the first rung and the second rung have equal strut lengths.

Example 142. The prosthetic valve of any example herein, particularly of any one of examples 132 to 141, wherein all angled struts of the third rung and the fourth rung have equal strut lengths.

Example 143. The prosthetic valve of any example herein, particularly of any one of examples 132 to 141, wherein each angled strut of the first rung has a strut width greater than a strut width of any angled strut of the third rung and the fourth rung.

Example 144. The prosthetic valve of any example herein, particularly of any one of examples 132 to 142, wherein each angled strut of the second rung has a strut width greater than a strut width of any angled strut of the third rung and the fourth rung.

Example 145. The prosthetic valve of any example herein, particularly of any one of examples 132 to 144, wherein the axial frame members comprise a plurality of axial struts and a plurality of commissure supports.

Example 146. The prosthetic valve of any example herein, particularly of example 145, wherein the commissure support is configured to support a commissure of a valvular structure that can be mounted in the frame.

Example 147. The prosthetic valve of any example herein, particularly of any one of examples 145 or 146, wherein each of the plurality of commissure supports comprises a commissure window.

Example 148. The prosthetic valve of any example herein, particularly of example 147, wherein the commissure window comprises a window opening between two window struts of the commissure window.

Example 149. The prosthetic valve of any example herein, particularly of any one of examples 147 or 148, wherein each commissure support further comprises an upper strut portion extending between the commissure window and the upper end of the commissure support.

Example 150. The prosthetic valve of any example herein, particularly of example 149, wherein each commissure support further comprises a lower strut portion extending between the commissure window and the lower end of the commissure support.

Example 151. The prosthetic valve of any example herein, particularly of any one of examples 147 or 148, wherein each commissure support further comprises a lower strut portion extending between the commissure window and the lower end of the commissure support.

Example 152. The prosthetic valve of any example herein, particularly of any one of examples 145 to 151, wherein a single one of the axial struts is disposed between immediately adjacent commissure supports.

Example 153. The prosthetic valve of any example herein, particularly of any one of examples 145 to 152, wherein a single one of the commissure supports is disposed between immediately adjacent axial struts.

Example 154. The prosthetic valve of any example herein, particularly of any one of examples 145 to 153, wherein the plurality of axial struts comprises three axial struts, and wherein the plurality of commissure supports comprises three commissure supports.

Example 155. The prosthetic valve of any example herein, particularly of any one of examples 145 to 154, wherein the axial struts and the commissure supports are alternately arranged around the circumference of the first cell row.

Example 156. The prosthetic valve of any example herein, particularly of any one of examples 132 to 155, wherein the frame further comprises a fifth rung comprising a plurality of angled struts, and a fourth cell row comprising a plurality of cells, each cell of the fourth cell row comprising two angled struts of the fourth rung and two angled struts of the fifth rung.

Example 157. The prosthetic valve of any example herein, particularly of example 156, wherein the frame further comprises a sixth rung comprising a plurality of angled struts, and a fifth cell row comprising a plurality of cells, each cell of the fifth cell row comprising two angled struts of the fifth rung and two angled struts of the sixth rung.

Example 158. The prosthetic valve of any example herein, particularly of any one of examples 132 to 157, further comprising a valvular structure mounted inside the frame and comprising a plurality of leaflets, each leaflet comprising a free edge closer to the first rung of the frame, and a cusp edge along which the leaflet is coupled to the frame.

Example 159. The prosthetic of any example herein, particularly of example 158, wherein an apex of the cusp edge is located at an inflow end of the frame.

Example 160. The prosthetic valve of any example herein, particularly of example 159, wherein the apex of the cusp edge is not connected to the frame.

Example 161. The prosthetic valve of any example herein, particularly of example 159, wherein the apex of the cusp edge is aligned with an inflow apex of the frame.

Example 162. The prosthetic valve of any example herein, particularly of any one of examples 158 to 161, wherein the plurality of leaflets comprises three leaflets.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising: a frame movable between a radially compressed state and a radially expanded state, the frame comprising: a first rung of angled struts interconnected at upper junctions and lower junctions of the first rung; a second rung of angled struts interconnected at upper junctions and lower junctions of the second rung, wherein the second rung is distal to the first rung; a plurality of axial frame members, each axial frame member connected at an upper end thereof to the first rung, and at a lower end thereof to the second rung; wherein the first rung comprises two angled struts extending between immediately adjacent axial frame members; and wherein the second rung comprises three angled struts extending between immediately adjacent axial frame members.

2. The prosthetic valve of claim 1, wherein the two angled struts extending between immediately adjacent axial frame members have different lengths.

3. The prosthetic valve of claim 1, wherein each upper junction of the first rung is circumferentially aligned with one of the lower junctions of the second rung.

4. The prosthetic valve of claim 1, wherein the axial frame members comprise a plurality of axial struts and a plurality of commissure supports, each commissure support comprising a commissure window.

5. The prosthetic valve of claim 4, wherein each commissure support is attached at its upper end to a lower junction of the first rung, and is attached at its lower end to a lower junction of the second rung.

6. The prosthetic valve of claim 4, wherein each axial strut is attached at its upper end to a lower junction of the first rung, and is attached at its lower end to an upper junction of the second rung.

7. The prosthetic valve of claim 1, wherein each upper junction of the first rung comprises an arcuate region defined between an upper curved surface and a lower curved surface thereof.

8. The prosthetic valve of claim 7, wherein a width of the arcuate region of each upper junction of the first rung is less than a width of an angled strut extending therefrom.

9. The prosthetic valve of claim 1, wherein each upper junction of the first rung is circumferentially aligned with a lower junction of the second rung.

10. The prosthetic valve of claim 1, wherein each angled strut of the first rung, extending between the upper end of the corresponding commissure support and the upper junction of the first rung, has a greater strut length than that of the angled strut of the first rung extending between the same upper junction of the first rung and the upper end of the corresponding axial strut.

11. The prosthetic valve of claim 1, further comprising free apices defined by the second rung between immediately adjacent axial frame members.

12. The prosthetic valve of claim 11, wherein the upper junctions of the first rung define outflow apices which are circumferentially offset from any of the free apices of the second rung.

13. The prosthetic valve of claim 11, wherein each free apex comprises an arcuate region defined between an upper curved surface and a lower curved surface thereof.

14. A prosthetic valve comprising: a frame movable between a radially compressed state and a radially expanded state, the frame comprising: a first rung of angled struts interconnected at upper junctions and lower junctions of the first rung; a second rung of angled struts interconnected at upper junctions and lower junctions of the second rung, wherein the second rung is distal to the first rung; a plurality of axial frame members, each axial frame member connected at an upper end thereof to the first rung, and at a lower end thereof to the second rung; wherein the first rung comprises two angled struts extending between immediately adjacent axial frame members; wherein the second rung comprises four angled struts extending between immediately adjacent axial frame members; and wherein each lower junction of the first rung is circumferentially aligned with one of the upper junctions of the second rung.

15. The prosthetic valve of claim 14, wherein the axial frame members comprise a plurality of axial struts and a plurality of commissure supports, each commissure support comprising a commissure window.

16. The prosthetic valve of claim 15, wherein each commissure support is attached at its upper end to an upper junction of the first rung, and is attached at its lower end to an upper junction of the second rung.

17. The prosthetic valve of claim 15, wherein each axial strut is attached at its upper end to an upper junction of the first rung, and is attached at its lower end to an upper junction of the second rung.

18. The prosthetic valve of claim 14, wherein each upper junction of the first rung comprises an arcuate region defining an upper curved surface opposite to the axial frame member attached to the upper junction of the first rung.

19. The prosthetic valve of claim 14, further comprising free apices defined by the second rung between immediately adjacent axial frame members.

20. The prosthetic valve of claim 19, wherein each lower junction of the first rung is circumferentially aligned with one of the free apices of the second rung.
